(19) United States
(12) Patent Application Publication
(10) Pub. No.: US 2020/0298233 A1
(43) Pub. Date: Sep. 24, 2020

(54) MICROFLUIDIC PAPER CHIP FOR DETECTING MICRO-ORGANISM, METHOD FOR PREPARING THE SAME AND METHOD FOR DETECTING MICRO-ORGANISM USING THE SAME

(71) Applicant: BIOMAX CO., LTD., Seoul (KR)

(72) Inventors: Chan Ho KWON, Namyangju-si, Gyeonggi-do (KR); Jae Hoon KIM, Seoul (KR)

(73) Assignee: BIOMAX CO., LTD., Seoul (KR)

(21) Appl. No.: 16/771,583

(22) PCT Filed: Dec. 17, 2018
(86) PCT No.: PCT/KR2018/016003

§ 371 Date: Jun. 10, 2020

(30) Foreign Application Priority Data
Dec. 19, 2017 (KR) 10-2017-0175505

(51) Int. Cl. B01L 3/00; C12Q 1/04

(52) U.S. Cl. CPC B01L 3/502761; C12Q 1/04; B01L 3/502707; B01L 2200/0647; C12Q 2304/20; B01L 2300/0887; B01L 2300/126; B01L 2300/165; B01L 2300/0819

(57) ABSTRACT

Disclosed is a microfluidic paper chip for detecting a microorganism, a method for preparing the same and a method for detecting the microorganism using the same. The microfluidic paper chip includes a lysis layer and a chromogenic layer which are laminated in this order. The lysis layer is composed of a hydrophilic paper containing a lysis reagent composition and the chromogenic layer is composed of a hydrophilic paper containing a chromogenic reagent.

### CROSS-REFERENCE TO PRIOR APPLICATIONS

This application is a National Stage Patent Application of PCT International Patent Application No. PCT/KR2018/016003 (filed on Dec. 17, 2018) under 35 U.S.C. 5371, which claims priority to Korean Patent Application No. 10-2017-0175505 (filed on Dec. 19, 2017), which are all hereby incorporated by reference in their entirety.

### BACKGROUND

The present invention relates to a microfluidic paper chip for detecting a microorganism, method for preparing the same and method for detecting the microorganism using the same. More specifically, the present invention relates to a microfluidic paper chip for detecting a microorganism in which a hydrophilic paper medium comprising a lysis reagent composition and a chromogenic reagent is sequentially laminated, method for preparing the same and method for detecting the microorganism using the same.

With an increasing demand of high food stability for food risk factors, a demand for rapid and accurate monitoring on food risk factors that may be generated during processes such as preparing, producing, and distributing food is rising. In particular, a rapid and accurate method for detecting food risk microorganisms that are capable of causing food poisoning is an essential technology for not only food safety, but also other various fields such as medical, health, and environment.

Current methods for detecting food risk microorganisms use various microorganism detecting-technologies ranging from detections using conventional microorganism media to PCR or immunological methods, and research methods for a quicker and more accurate detection is being developed through new technologies such as DNA chip, microfluidics, DNA array.

A technology commonly used in food risk microorganism detection is a conventional culture method that uses a selective medium for each food risk microorganism, which requires culture time in enrichment culture and selective medium and exhibits a disadvantage of requiring inconvenient work and manpower.

To reduce the time and inconvenient work, technologies including PCR, DNA chip, various microfluidics and DNA array have been developed or are under development. These technologies, however, exhibit disadvantages of requiring expensive instruments or reagents, and specialized technology and knowledge for detection.

As the importance of developing on-site detection technologies to compensate the disadvantages of such specialized detection technology was suggested, ATP assay and antibody-based immunological detection method were developed. However, the ATP assay is a convenient method with high sensitivity while being incapable of analyzing specificity; the immunological detection method has high specificity while the sensitivity is low, and because the method uses antibody, the method expresses disadvantages of high price, limited product storage and distribution, etc.

Therefore, a product that is capable of an economical monitoring in food risk microorganism detection sites while having high specificity and sensitivity and capable of being stored and distributed at room temperature with low detection cost is needed as an on-site detection technology, and demand for multiple detections is rising as different food risk microorganism detections are attempted for various samples in the food risk microorganism detection sites. However, currently a product capable of performing multiple detections as an on-site detection technology does not exist.

### SUMMARY

In order to solve the aforementioned problem, the present invention is to provide a microfluidic paper chip for a paper-based microorganism detection that uses a chromogenic substrate that reacts with a specific enzyme of a microorganism in detecting the microorganism, allows an easy and quick detection of microorganisms through a unique coloring and is capable of detecting the microorganism efficiently in a small space at a small cost.

In order to solve the aforementioned problem, the present invention provides a microfluidic paper chip for detecting a microorganism in which a lysis layer formed with a paper made of a hydrophilic material comprising a lysis reagent composition and a chromogenic layer formed with a paper made of a hydrophilic material comprising a chromogenic reagent are sequentially laminated.

Further, the present invention provides a microfluidic paper chip for detecting a microorganism that is characterized by having an outer layer formed with a paper made of a hydrophilic material additionally laminated over the lysis layer or under the chromogenic layer.

Further, the present invention provides a microfluidic paper chip for detecting a microorganism that is characterized by having an oxidation layer formed with a paper made of a hydrophilic material comprising an oxidation reagent additionally laminated between the lysis layer and the chromogenic layer.

Further, the present invention provides a microfluidic paper chip for detecting a microorganism that is characterized by forming a fluidic channel by printing a hydrophobic material on the edges of said paper made of a hydrophilic material and forming a wall.

Further, the present invention provides a microfluidic paper chip for detecting a microorganism that is characterized by that the paper made of a hydrophilic material is a chromatography paper or a filter paper.

Further, the present invention provides a microfluidic paper chip that is characterized by that the microorganism is at least one selected from the group consisting of Salmonella, Bacillus, Listeria, Vibrio, Campylobacter, Staphylococcus aureus, Escherichia Coliform, E. coli, Shigella, Legionella, Enterobacter sakazakii, Citrobacter, Proteus, Methicillin-resistant Staphylococcus aureus (MRSA), and E. coli O157.

Further, the present invention provides a microfluidic paper chip that is characterized by that the lysis reagent composition is at least one selected from the group consisting of Tergitol NP-9, Tergitol NP-10, Tergitol NP-40, Triton X-100, Tween 80, BMT, SB3-8, SB3-10, SB3-14, and SB3-16.

Further, the present invention provides a microfluidic paper chip that is characterized by that the lysis reagent composition additionally comprises C7BzO (3-[[3-(4-heptylphenyl)-3-hydroxypropyl]-dimethylazaniumyl]propane-1-sulfonate).

Further, the present invention provides a microfluidic paper chip that is characterized by that the lysis reagent composition additionally comprises a silica bead.

Further, the present invention provides a microfluidic paper chip that is characterized by that the chromogenic reagent composition is at least one selected from the group consisting of 5-bromo-4-chloro-3-indoxyl-beta-L-arabinopyranoside, 5-bromo-4-chloro-3-indoxyl-beta-D-glucuronic acid, 5-bromo-4-Chloro-3-indoxyl-alpha-D-maltotrioside, 5-bromo-4-chloro-3-indoxyl-N-acetyl-beta-D-galactosamide, 5-bromo-4-Chloro-3-indoxyl-N-acetyl-beta-D-glucosaminid, 5-bromo-4-chloro-3-indoxyl-N-acetyl-beta-D-galactosamide, 5-Bromo-4-chloro-3-indoxyl-alpha-D-N-acetylneuraminic acid, 5-bromo-4-chloro-3-indoxyl-alpha-L-araminofuranoside, 5-bromo-4-Chloro-3-indoxyl-beta-D-cellobioside, 5-bromo-4-chloro-3-indoxyl-choline phosphate, 5-bromo-4-chloro-3-indoxyl-alpha-D-fucopyranoside, 5-bromo-4-chloro-3-indoxyl-alpha-L-fucoparinoside, 5-bromo-4-chloro-3-indoxyl-alpha-D-galactopyranoside, 5-bromo-4-chloro-3-indoxyl-beta-D-galactopyranoside, 5-bromo-4-chloro-3-indoxyl-alpha-D-glucopyranoside, 5-bromo-4-chloro-3-indoxyl-beta-D-glucopyranoside, 5-bromo-4-chloro-3-indoxyl-myo-inositol-1-phosphate, 5-bromo-4-chloro-3-indoxyl-alpha-D-mannopyranoside, 5-bromo-4-chloro-3-indoxyl-beta-D-mannopyranoside, 5-bromo-4-chloro-3-indoxyl-alpha-D-xylopyranoside, 5-bromo-4-chloro-3-indoxyl butylate, 5-bromo-4-chloro-3-indoxyl caprylate, 5-Bromo-4-chloro-3-indoxyl nonanonate, 5-bromo-4-chloro-3-indoxyl oleate, 5-bromo-4-chloro-3-indoxyl palmitate, 5-Bromo-4-chloro-3-indoxyl phosphate, 5-bromo-4-chloro-3-indoxyl sulfate, 5-bromo-4-chloro-3-indoxyl-1-acetate, 5-bromo-4-chloro-3-indoxyl-3-acetate, 6-chloro-3-indoxyl-N-acetyl-beta-D-glucosaminide, 6-chloro-3-indoxyl-alpha-D-mannopyranoside, 6-chloro-3-indoxyl-beta-D-mannopyranoside, 6-chloro-3-Indoxyl-myo-inositol-1-phosphate, 6-chloro-3-indoxyl-N-acetyl-beta-D-galactosaminide, 6-chloro-3-indoxyl-beta-D-cellobioside, 6-chloro-3-indoxyl-alpha-D-galactopyranoside, 6-chloro-3-indoxyl-beta-D-galactopyranoside, 6-chloro-3-indoxyl-alpha-D-glucopyranoside, 6-chloro-3-indoxyl-beta-D-glucopyranoside, 6-chloro-3-indoxyl-beta-D-glucuronic acid, 6-chloro-3-indoxyl butylate, 6-Chloro-3-indoxyl caprylate, 6-Chloro-3-indoxyl nonanoate, 6-Chloro-3-indoxyl oleate, 6-Chloro-3-indoxyl palmitate, 6-chloro-3-indoxyl phosphate, 6-chloro-3-indoxyl sulfate, 6-chloro-3-indoxyl-1-acetate, 5-bromo-6-chloro-3-indoxyl-N-acetyl-beta-D-glucosaminide, 5-bromo-6-chloro-3-indoxyl-beta-D-fucopyranoside, 5-bromo-6-chloro-3-indoxyl-alpha-D-galactopyranoside, 5-bromo-6-chloro-3-indoxyl-beta-D-galactopyranoside, 5-bromo-6-chloro-3-indoxyl-alpha-D-glucopyranoside, 5-bromo-6-chloro-3-indoxyl-beta-D-glucuronic acid, 5-bromo-6-chloro-3-indoxyl-alpha-D-glucopyranoside, 5-bromo-6-chloro-3-indoxyl-myo-inositol-1-phosphate, 5-bromo-6-chloro-3-indoxyl butylate, 5-bromo-6-chloro-3-indoxyl caprylate, 5-bromo-6-chloro-3-indoxyl nonanonate, 5-bromo-6-chloro-3-indoxyl palmitate, 5-bromo-6-chloro-3-indoxyl choline phosphate, 5-bromo-6-chloro-3-indoxyl phosphate, 5-bromo-6-chloro-3-indoxyl sulfate, 5-bromo-6-chloro-3-indoxyl-3-acetate, Aldol 518 beta-D-galactopyranoside, Aldol 518 alpha-D-galactopyranoside, Aldol 518 alpha-D-glucopyranoside, Aldol 518 beta-D-glucopyranoside, Aldol 518 beta-D-glucuronic acid, Aldol 518 myo-inositol-1-phosphate, Aldol 515 caprylate, Aldol 515 palmitate, Aldol 515 phosphate and Aldol 515 acetate.

Further, the present invention provides a microfluidic paper chip that is characterized by that the oxidation reagent is at least one selected from the group consisting of a mixture of potassium ferriccyanide (K₃Fe(CN)₆) and potassium ferrocyanide (K₄Fe(CN)₆), a mixture of FeCl₂ and FeCl₃, and a mixture of FeSO₄ and FeCl₂.

Further, the present invention provides a method for preparing microfluidic paper chip that comprises: (a) a step of forming a hydrophobic wall by printing a hydrophobic material on the edges of multiple papers that are formed with a hydrophilic material; (b) a step of absorbing a lysis reagent composition to a hydrophilic area of a paper on which said hydrophobic material is printed and drying; (c) a step of absorbing a chromogenic reagent to a hydrophilic area of another paper on which said hydrophobic material is printed and drying; (d) a step of sequentially laminating the paper on which said hydrophobic material is printed—the paper on which said lysis reagent composition is absorbed—the paper on which said chromogenic reagent is abasorbed—the paper on which said hydrophobic material is printed.

Further, the present invention provides a method of detecting a microorganism by using said microfluidic paper chip.

By using the microfluidic paper chip of the present invention, an easy and quick detection of a microorganism is possible through a unique coloring by using a chromogenic substrate that reacts with a specific enzyme of the microorganism, and detecting the microorganism efficiently in a small space at a small cost is also possible.

### BRIEF DESCRIPTION OF THE DRAWINGS

FIG. 1 is photographs of SDS-PAGE to identify lysis efficacy of 5 food risk microorganisms for different types of lysis reagents,

FIG. 2 is a graph showing the results of lysis efficacy of 5 food risk microorganisms for different types of lysis reagents measured with BCA assay,

FIG. 3 is a diagram that observed the coloring level of Vibrio vulnificus for different types of chromogenic reagent,

FIG. 4 is a diagram that observed the coloring level of Salmonella spp. for different types of chromogenic reagent,

FIG. 5 is a photograph of coloring reaction test results of Magenta-caprylate for different types of food risk microorganisms,

FIG. 6 is a diagram that observed the coloring level of Escherichia coli O157 for different types of chromogenic reagent,

FIG. 7 is a diagram that observed the coloring level of Escherichia coli for different types of chromogenic reagent,

FIG. 8 is a diagram that observed the coloring level of Listeria monocytogenes for different types of chromogenic reagent,

FIG. 9 is a diagram that observed the coloring level of Staphylococcus aureus for different types of chromogenic reagent,

FIG. 10 is photographs of coloring reaction test results of Magenta-beta-galactopyranoside for different concentrations of oxidation reagent,

FIG. 11 is photographs of coloring reaction test results of X-beta-glucopyranoside for different concentrations of oxidation reagent,

FIG. 12 is photographs of coloring reaction test results of X-phosphate for different concentrations of oxidation reagent,

FIG. 13 is a photograph of coloring reaction test results of Magenta-caprylate for different concentrations of oxidation reagent,

FIG. 14 is a photograph of coloring reaction test results of X-beta-glucuronide for different concentrations of oxidation reagent,

FIG. 15 is a photograph of coloring reaction test results of Aldol-myo-inositol-1-phosphate for different concentrations of oxidation reagent,

FIG. 16 is photographs of coloring reaction test results of Magenta-beta-galactopyranoside for different types and concentrations of oxidation reagent in detection of Vibrio vulnificus,

FIG. 17 is photographs of coloring reaction test results of Magenta-caprylate for different types and concentrations of oxidation reagent in detection of Salmonella spp.,

FIG. 18 is photographs of coloring reaction test results of X-phosphate for different types and concentrations of oxidation reagent in detection of Staphylococcus aureus,

FIG. 19 is photographs of coloring reaction test results of Aldol-myo-inositol phosphate for different types and concentrations of oxidation reagent in detection of Listeria monocytogenes,

FIG. 20 is photographs of coloring reaction test results of Magenta-beta-galactopyranoside for different types and concentrations of oxidation reagent in detection of Escherichia coli O157,

FIG. 21 is a diagram showing an example of a sketch for a paper medium prepared by printing with a wax print (the black area of the sketch represents the hydrophobic area with wax coating, and the white area of the diagram shows the hydrophilic area with no wax coating),

FIG. 22 is a diagram showing composition parts (A), assembly steps (B), and appearance of the chip after completion of assembly (C) for preparing a microfluidic paper chip,

FIG. 23 is photographs of coloring reaction test results for different paper thicknesses (Top: Detection of Escherichia coli O157, Bottom: Detection of Staphylococcus aureus),

FIG. 24 is photographs of coloring reaction test results for different paper pore sizes (Top: Detection of Escherichia coli O157, Bottom: Detection of Staphylococcus aureus),

FIG. 25 is photographs of coloring reaction test results for different sizes of hydrophilic areas in paper media (Top: Detection of Escherichia coli O157, Bottom: Detection of Staphylococcus aureus),

FIG. 26 is photographs of coloring reaction test results of Escherichia coli for different types and concentrations of oxidation reagents,

FIG. 27 is photographs of coloring reaction test results of Escherichia coli O157 for different types and concentrations of oxidation reagents,

FIG. 28 is photographs of coloring reaction test results of Escherichia coli O157 for different concentrations of Magenta-beta-galactopyranoside,

FIG. 29 is photographs of coloring reaction test results of Escherichia coli for different concentrations of X-beta-glucuronide,

FIG. 30 is photographs of coloring reaction test results of Escherichia coli O157 for different concentrations of Magenta-beta-galactopyranoside to 0.1 M X-beta-glucuronide,

FIG. 31 is photographs of coloring reaction test results of Escherichia coli for different concentrations of Magenta-beta-galactopyranoside to 0.1 M X-beta-glucuronide,

FIG. 32 is photographs of coloring reaction test results of paper-based microfluidic device for detecting Escherichia coli O157,

FIG. 33 is photographs of coloring reaction test results of Vibrio vulnificus for different types and concentrations of oxidation reagents,

FIG. 34 is photographs of coloring reaction test results of Vibrio vulnificus for different concentrations of X-beta-glucopyranoside,

FIG. 35 is photos of coloring reaction test results of paper-based microfluidic device for detecting Vibrio vulnificus,

FIG. 36 is photographs of Salmone-alpha-glucopyranoside coloring reaction test results of Salmonella spp. for different types and concentrations of oxidation reagents,

FIG. 37 is photographs of coloring reaction test results of Salmonella spp. for different concentrations of Salmone-alpha-glucopyranoside,

FIG. 38 is photographs of coloring reaction test results of Salmonella spp. for different concentrations of X-phosphate,

FIG. 39 is photographs of coloring reaction test results of Salmonella spp. for different concentrations of X-phosphate to 0.2 M Salmone-alpha-glucopyranoside,

FIG. 40 is photographs of coloring reaction test results of paper-based microfluidic device for detecting Salmonella spp.,

FIG. 41 is photographs of coloring reaction test results of Listeria monocytogenes for different types and concentrations of oxidation agents to Aldol-myo-inositol-phosphate,

FIG. 42 is photographs of coloring reaction test results of Listeria monocytogenes for different concentrations of Aldol-myo-inositol-phosphate,

FIG. 43 is photographs of coloring reaction test results of Listeria monocytogenes for different concentrations of Aldol-myo-inositol-phosphate,

FIG. 44 is photographs of coloring reaction test results of paper-based microfluidic device for detecting Listeria monocytogenes,

FIG. 45 is photographs of coloring reaction test results of Staphylococcus aureus for different types and concentrations of oxidation agents to X-phosphate,

FIG. 46 is photographs of coloring reaction test results of Staphylococcus aureus for different concentrations of Magenta-beta-galactopyranoside,

FIG. 47 is photographs of coloring reaction test results of Staphylococcus aureus for different concentrations of X-phosphate,

FIG. 48 is photographs of coloring reaction test results of Staphylococcus aureus for different concentrations of X-phosphate to 0.1 M Magenta-beta-galactopyranoside,

FIG. 49 is photographs of coloring reaction test results of paper-based microfluidic device for detecting Staphylococcus aureus.

### DETAILED DESCRIPTION

Hereinafter, the present invention will be described in detail according to preferable embodiments. Prior to this, terms or words used in the present specification and claims shall not be interpreted as being limited to typical or dictionary meanings, but should be interpreted as a meaning and a concept that is consistent with the context of the technical spirit of the invention, based on the principle that an inventor may properly define the meaning of the terms to best explain the invention. Accordingly, since the embodiments set forth in the present specification are just the most preferred embodiment of the present invention but do not represent all the technological spirit of the present invention, it should be understood that embodiments of the present invention are capable of various modifications, equivalents, and alternatives at the time of present application.

The present invention discloses a microfluidic paper chip for detecting a microorganism in which a lysis layer formed with a paper made of a hydrophilic material comprising a lysis reagent composition and a chromogenic layer formed with a paper made of a hydrophilic material comprising a chromogenic reagent are sequentially laminated.

The microfluidic paper chip for detecting a microorganism provided by the present invention is a device that is capable of identifying the existence of a microorganism of interest in a sample subject to the detection by simply injecting the sample subject to the detection. More specifically, a lysis reaction of a microorganism is carried out by a lysis reagent comprised in the lysis layer when the sample subject to the detection is injected to the microfluidic paper chip for detecting a microorganism, a specific chromogenic reagent comprised in the coloring part reacts with an enzyme existing in the microorganism subject to the detection, carries out a coloring reaction, and the result is expressed.

In the present invention, an outer layer formed with a paper made of a hydrophilic material may be additionally laminated over the lysis layer or under the chromogenic layer. By additionally laminating the outer layer, a fine space in which a reaction carries out is secured and the reaction may be more stable, and the lysis layer or the chromogenic layer may be protected from being contaminated by a foreign material.

In the present invention, the type of the paper is not particularly limited as long as the paper is formed with a hydrophilic material, and preferably a chromatography paper or a filter paper may be used.

Although the thickness of the paper is not particularly limited, the thickness may be 100-1000 μm for a stable coloring reaction, preferably be 200-500 μm, and most preferably 300-500 μm. If the thickness of the paper is less than 100 μm, a sufficient space in which an enzyme existing in a microorganism and a chromogenic reagent reacts to carry out a coloring reaction may not be provided, and if the thickness exceeds 1000 μm, the thickness of the chip may be too thick and increase the used amount of reagent, and undesirably long time may be required to express the detection result.

In the present invention, the paper is preferable to be a porous paper, wherein the pore size of the paper may be 3-30 μm, preferably 5-30 μm, and most preferably 7-25 μm.

In the present invention, the paper formed with a hydrophilic material may have formed a fluidic channel by forming a wall from printing a hydrophobic material on the edges. In the present invention, the type of the hydrophobic material is not specifically limited as long as the material is capable of controlling the diffusion of water-soluble fluids by being printed on a paper of a hydrophilic material, preferably be a hydrophobic element such as a wax or a photosensitive polymer, and most preferably a wax.

The microfluidic paper chip of the present invention must have a consistent flow of fluid that penetrates the top and the bottom of the paper chip, since the existence of the microorganism of interest can be identified via a coloring reaction during the process in which an injected sample subject to the detection transfers by being sequentially absorbed to the lysis layer and the chromogenic layer. Thus, the papers of the hydrophilic material that constitute the lysis layer, the chromogenic layer and the outer layer may each have the edges coated with a hydrophobic material such as a wax or a photosensitive polymer and be formed into a hydrophobic area with the exception of hydrophilic areas of an identical shape, thereby have the injected sample subject to the detection not absorbed and spread into peripheral areas of each layer and be capable of being transferred sequentially to each layer with ease.

In the present invention, the outer layer is a layer that serves as an inlet in which the sample subject to the detection is injected, and the paper of a hydrophilic material with wax-coated edges can be used.

In the present invention, the paper layer made of a hydrophilic material comprises a lysis reagent composition, and lysis phenomenon of the microorganisms existing in the sample subject to the detection injected into the lysis layer is induced in the layer.

In the present invention, the lysis reagent composition comprised in the lysis layer is not limited as long as the composition is a lysis buffer commonly used in the art, and preferably a composition comprising a surfactant, a cationic detergent, an anionic detergent, and a nonionic detergent may be used. In the present invention, non-limiting examples of the surfactant and the detergents are Tergitol NP-9, Tergitol NP-10, Tergitol NP-40, Triton X-100, Tween 80, BMT, SB3-8, SB3-10, SB3-14, SB3-16, etc.

In the present invention, the chromogenic layer comprises a chromogenic reagent for a microorganism-unique enzyme comprised by the microorganism lysed in the lysis layer, and carries out a unique coloring reaction when the microorganism of interest exists in the sample subject to the detection.

Thus, the type of microorganism subject to the detection is not specifically limited in the microfluidic paper chip for detecting a microorganism of the present invention, and the type of microorganism that can be detected by using the microfluidic paper chip according to the present invention is not limited if a chromogenic reagent capable of carrying out a coloring reaction specific to a unique enzyme existing in a microorganism is appropriately selected and applied to the third layer.

For the chromogenic reagent used in this case, a chromogenic reagent unique to two types of target enzymes dominantly existing in a microorganism can be used, wherein the chromogenic reagent consists of a chromophore that expresses coloring and a unique substrate and expresses a unique color when incised by an enzyme existing in a microorganism. Chromophores that appear after incised by an enzyme express unique colors, such as yellow, red, blue, purple, and so on, and here, a combination can be made to enable detection of each microorganism through a cross validation with two enzymes, and various microorganisms can be detected under categorization through unique colors produced accordingly.

For example, in case of Listeria monocytogenes, 5-Bromo-4-chloro-3-indolyl-myo-inositol-1-phosphate, a chromogenic reagent that shows blue color, and 5-Bromo-6-chloro-3-indolyl-beta-D-glucopyranoside, a reagent that shows red color, are used to show purple color when Listeria monocytogenes is detected, and qualitative and quantitative detections are possible, since the color increases in accordance with the concentration of the detected microorganism.

The chromogenic reagent can be changed in case an enzyme of interest is overlapped among the multiple microorganisms to be detected, thus the chromogenic reagent can be constituted to prevent any error from crossing. For example, beta-glucosidase, a target enzyme of Listeria monocytogenes, is also a target enzyme of Vibrio vulnificus, thus 5-Bromo-6-chloro-3-indolyl-beta-D-glucopyranoside, a reagent that shows red color can be used, and for Vibrio vulnificus, Aldol 484 beta-D-glucopyranoside, a reagent that shows orange color can be used to distinguish the detections by not only the difference from other cross-supplemented enzymes but also the difference in coloring.

Further, not only a qualitative analysis but also a quantitative analysis from coloring reaction is possible by the microfluidic paper chip of the present invention, and specifically, a quantitative analysis is possible by analyzing and standardizing the difference of color level according to the number of microorganism.

Preferably, the chromogenic reagent in the present invention is at least one selected from the group consisting of 5-bromo-4-chloro-3-indoxyl-beta-L-arabinopyranoside, 5-bromo-4-chloro-3-indoxyl-beta-D-glucuronic acid, 5-bromo-4-Chloro-3-indoxyl-alpha-D-maltotrioside, 5-bromo-4-chloro-3-indoxyl-N-acetyl-beta-D-galactosamide, 5-bromo-4-Chloro-3-indoxyl-N-acetyl-beta-D-glucosaminid, 5-bromo-4-chloro-3-indoxyl-N-acetyl-beta-D-galactosamide, 5-Bromo-4-chloro-3-indoxyl-alpha-D-N-acetylneuraminic acid, 5-bromo-4-chloro-3-indoxyl-alpha-L-araminofuranoside, 5-bromo-4-Chloro-3-indoxyl-beta-D-cellobioside, 5-bromo-4-chloro-3-indoxyl-choline phosphate, 5-bromo-4-chloro-3-indoxyl-alpha-D-fucopyranoside, 5-bromo-4-chloro-3-indoxyl-alpha-L-fucoparinoside, 5-bromo-4-chloro-3-indoxyl-alpha-D-galactopyranoside, 5-bromo-4-chloro-3-indoxyl-beta-D-galactopyranoside, 5-bromo-4-chloro-3-indoxyl-alpha-D-glucopyranoside, 5-bromo-4-chloro-3-indoxyl-beta-D-glucopyranoside, 5-bromo-4-chloro-3-indoxyl-myo-inositol-1-phosphate, 5-bromo-4-chloro-3-indoxyl-alpha-D-mannopyranoside, 5-bromo-4-chloro-3-indoxyl-beta-D-mannopyranoside, 5-bromo-4-chloro-3-indoxyl-alpha-D-xylopyranoside, 5-bromo-4-chloro-3-indoxyl butylate, 5-bromo-4-chloro-3-indoxyl caprylate, 5-Bromo-4-chloro-3-indoxyl nonanonate, 5-bromo-4-chloro-3-indoxyl oleate, 5-bromo-4-chloro-3-indoxyl palmitate, 5-Bromo-4-chloro-3-indoxyl phosphate, 5-bromo-4-chloro-3-indoxyl sulfate, 5-bromo-4-chloro-3-indoxyl-1-acetate, 5-bromo-4-chloro-3-indoxyl-3-acetate, 6-chloro-3-indoxyl-N-acetyl-beta-D-glucosaminide, 6-chloro-3-indoxyl-alpha-D-mannopyranoside, 6-chloro-3-indoxyl-beta-D-mannopyranoside, 6-chloro-3-Indoxyl-myo-inositol-1-phosphate, 6-chloro-3-indoxyl-N-acetyl-beta-D-galactosaminide, 6-chloro-3-indoxyl-beta-D-cellobioside, 6-chloro-3-indoxyl-alpha-D-galactopyranoside, 6-chloro-3-indoxyl-beta-D-galactopyranoside, 6-chloro-3-indoxyl-alpha-D-glucopyranoside, 6-chloro-3-indoxyl-beta-D-glucopyranoside, 6-chloro-3-indoxyl-beta-D-glucuronic acid, 6-chloro-3-indoxyl butylate, 6-Chloro-3-indoxyl caprylate, 6-Chloro-3-indoxyl nonanoate, 6-Chloro-3-indoxyl oleate, 6-Chloro-3-indoxyl palmitate, 6-chloro-3-indoxyl phosphate, 6-chloro-3-indoxyl sulfate, 6-chloro-3-indoxyl-1-acetate, 5-bromo-6-chloro-3-indoxyl-N-acetyl-beta-D-glucosaminide, 5-bromo-6-chloro-3-indoxyl-beta-D-fucopyranoside, 5-bromo-6-chloro-3-indoxyl-alpha-D-galactopyranoside, 5-bromo-6-chloro-3-indoxyl-beta-D-galactopyranoside, 5-bromo-6-chloro-3-indoxyl-alpha-D-glucopyranoside, 5-bromo-6-chloro-3-indoxyl-beta-D-glucuronic acid, 5-bromo-6-chloro-3-indoxyl-alpha-D-glucopyranoside, 5-bromo-6-chloro-3-indoxyl-myo-inositol-1-phosphate, 5-bromo-6-chloro-3-indoxyl butylate, 5-bromo-6-chloro-3-indoxyl caprylate, 5-bromo-6-chloro-3-indoxyl nonanonate, 5-bromo-6-chloro-3-indoxyl palmitate, 5-bromo-6-chloro-3-indoxyl choline phosphate, 5-bromo-6-chloro-3-indoxyl phosphate, 5-bromo-6-chloro-3-indoxyl sulfate, 5-bromo-6-chloro-3-indoxyl-3-acetate, Aldol 518 beta-D-galactopyranoside, Aldol 518 alpha-D-galactopyranoside, Aldol 518 alpha-D-glucopyranoside, Aldol 518 beta-D-glucopyranoside, Aldol 518 beta-D-glucuronic acid, Aldol 518 myo-inositol-1-phosphate, Aldol 515 caprylate, Aldol 515 palmitate, Aldol 515 phosphate and Aldol 515 acetate, and the microorganism that can be detected is a food risk microorganism, at least one selected from the group consisting of Salmonella, Bacillus, Listeria, Vibrio, Campylobacter, Staphylococcus aureus, Escherichia Coliform, E. coli, Shigella, Legionella, Enterobacter sakazakii, Citrobacter, Proteus, Methicillin-resistant Staphylococcus aureus (MRSA), and E. coli O157.

The microfluidic paper chip of the present invention may have a paper formed with a hydrophilic material that comprises an oxidation reagent additionally laminated between the second layer and the third layer.

The oxidation reagent may serve a role of increasing the detection speed by stimulating the chromophore oxidation of the chromogenic reagent during the microorganism detection.

In the present invention, the outer layer that is formed with a paper of hydrophilic material under the chromogenic layer is a layer in which a coloring phenomenon induced by the enzyme-chromogenic reagent reaction in the chromogenic layer is applied, and just as the outer layer that is formed with a paper of hydrophilic material on the lysis layer, the paper of a hydrophilic material with wax-coated edges can be used.

The microfluidic paper chip of the present invention may comprise a cast that is capable of fixing and binding the lysis layer and the chromogenic layer after lamination. The top surface of the cast may have formed a hole to which a sample subject to the detection can be injected, and the bottom surface of the cast may have formed a hole from which the matter of chromogenic reaction can be observed.

The present invention further provides a method for preparing a microfluidic paper chip that comprises: (a) a step of forming a hydrophobic wall by printing a hydrophobic material on the edges of multiple papers that are formed with a hydrophilic material; (b) a step of absorbing a lysis reagent composition to a hydrophilic area of a paper on which said hydrophobic material is printed and drying; (c) a step of absorbing a chromogenic reagent to a hydrophilic area of another paper on which said hydrophobic material is printed and drying; (d) a step of sequentially laminating the paper on which said hydrophobic material is printed—the paper on which said lysis reagent composition is absorbed—the paper on which said chromogenic reagent is absorbed—the paper on which said hydrophobic material is printed.

The present invention further provides a method for detecting a microorganism by using said microfluidic paper chip.

Hereinafter, the present invention will be explained in detail with reference to examples.

### Example 1

Distribution and Culture of Microorganism

To utilize a type strain for detecting food risk microorganisms, registration was submitted to a domestic microorganism distributing organization and the type strain was received. The received food risk microorganisms and the organization of distribution are listed in the table 1 below, and the culture media of each microorganism are listed in the table 2 below.

TABLE 1

| Tested Strain name | Culture No. | Reference |
|---|---|---|
| Salmonella spp. | CCRM0119 | Culture Collection of |
| | | Antibiotics Resistant |
| | | Microbes |
| Escherichia | B3445 | Helicobacter pylori |
| coli (O157) | | Korean Type Culture |
| | | Collection |
| Listeria | KCTC3569 | Korean Collection for |
| monocytogenes | | Type Cultures |
| Staphylococcus | KCTC3881 | Korean Collection for |
| aureus | | Type Cultures |
| Vibrio vulnificus | KCTC2959 | Korean Collection for |
| | | Type Cultures |

TABLE 2

| Tested Strain | Solid | | |
|---|---|---|---|
| name | media | Broth media | Selective additives |
| Salmonella | XLD agar | RV broth | |
| Staphylococcus | Baird | TSB + Licl (5 mg) | EggYork (50 ml) |
| | Parker | | |
| | Agar | | |
| Vibrio | TCBS Agar | TSB + Nacl (5 g) | |
| Listeria | Oxford | Listeria | acriflavin (5 mg) |
| | Listeria | Enrichment Broth | cefotetan (2 mg) |
| | Agar | | colistin (20 mg) |
| | | | phophomycin |
| | | | (10 mg) |
| E. coli | MacConkey | TSB + Novobiocin | |
| O157:H7 | Aaar with | (20 mg) | |
| | Sorbitol | | |

### Example 2

Development of a Lysis Reagent Composition for Detecting Microorganisms

To search for an effective lysis reagent for the 5 food risk microorganisms cultured in the example 1, bacterial lysis efficacy for various detergents was tested by measuring optical density (OD).

To search for a lysis reagent, sodium dodecyl sulfate (SDS), an anion detergent, 5 types of nonionic detergents including Tergitol NP-9, 5 types of cation detergents including Tween 80, and 5 types of bipolar detergents including 3-[Dimethyl(n-octyl)ammonio]propane-1-sulfonate were tested, which are 16 detergents in total (data not shown).

(1) Bacterial Lysis Efficacy Test for Different Lysis Detergent Types

Among the detergents, bacterial lysis efficacy for SDS, Tergitol NP-9, Tergitol NP-10, Tergitol NP-40, Triton X-100, 1-Butyl-3-methylimidazolium Thiocyanate (BMT), Tween 80, 3-[Dimethyl(n-octyl)ammonio]propane-1-sulfonate (SB3-8), 3-(Dodecyldimethylammonio)propane-1-sulfonate (SB3-10), 3-[Dimethyl (tetradecyl)ammonio]propane-1-sulfonate (SB3-14), and 3-(Hexadecyldimethyl ammonio)propane-1-sulfonate (SB3-16), which are 10 types of detergents that were found to have excellent lysis efficacy, was tested by measuring optical density.

Specifically, after pre-culturing the 5 types of food risk microorganisms in separate liquid culture media, the microorganisms were inoculated at inoculum volume of 1% (v/v), cultured for 24 hours, the OD of the culture medium was measured and diluted with phosphate salt buffer (PSB) to set the OD value to be approximately at 1.5, the 10 types of detergents were added to be 1% and the OD value was measured after 10 minutes to test the lysis efficacy for different types of lysis reagents, and the results were listed in the table 3 and table 4 below.

TABLE 3

| Tested Strain | Detergent (1%) | Optical Density (O.D.) |
|---|---|---|
| Salmonella spp. | No addition | 1.5916/1.5921/1.5928 |
| | Tergitol NP-9 | 1.8587/1.8606/1.8573 |
| | Tergitol NP-10 | 1.8659/1.8616/1.8644 |
| | Tergitol NP-40 | 1.8424/1.8423/1.8424 |
| | Triton X-100 | 1.2843/1.2957/1.3038 |
| | BMT | 1.5816/1.5874/1.5828 |
| | Tween 80 | 1.7399/1.7444/1.7390 |
| | SB3-8 | 1.5322/1.5382/1.5442 |
| | SB3-10 | 0.3379/0.3407/0.3476 |
| | SB3-14 | 0.3477/0.3488/0.3494 |
| | SB3-16 | 0.3597/0.3591/0.3604 |
| Vibrio | No addition | 1.6746/1.6726/1.6744 |
| vulnificus | Tergitol NP-9 | 0.4970/0.4905/0.4945 |
| | Tergitol NP-10 | 0.4982/0.5000/0.5017 |
| | Tergitol NP-40 | 0.4826/0.4780/0.4773 |
| | Triton X-100 | 0.5025/0.4646/0.4499 |
| | BMT | 1.7676/1.7762/1.7759 |
| | Tween 80 | 1.7242/1.7219/1.7191 |
| | SB3-8 | 1.7632/1.7561/1.7753 |
| | SB3-10 | 0.2252/0.2200/0.2172 |
| | SB3-14 | 0.0845/0.0845/0.0832 |
| | SB3-16 | 0.0675/0.0691/0.0653 |

TABLE 4

| Tested Strain | Detergent (1%) | Optical Density (O.D.) |
|---|---|---|
| Esherichia | No addition | 1.5221/1.5193/1.5282 |
| coli O157 | Tergitol NP-9 | 1.3663/1.3810/1.3519 |
| | Tergitol NP-10 | 1.3652/1.2682/1.1693 |
| | Tergitol NP-40 | 1.4858/1.4820/1.5095 |
| | Triton X-100 | 1.0816/1.1533/1.1301 |
| | BMT | 1.5454/1.5652/1.5713 |
| | Tween 80 | 1.5667/1.5885/1.5893 |
| | SB3-8 | 1.4158/1.4512/1.4301 |
| | SB3-10 | 0.7586/0.7513/0.7550 |
| | SB3-14 | 0.3954/0.3883/0.3928 |
| | SB3-16 | 0.7902/0.7852/0.7697 |
| Listeria | No addition | 1.7053/1.6844/1.6903 |
| monocytogenes | Tergitol NP-9 | 1.6332/1.6236/1.6121 |
| | Tergitol NP-10 | 1.4746/1.4691/1.4729 |
| | Tergitol NP-40 | 1.1091/1.1483/1.1675 |
| | Triton X-100 | 1.4816/1.4820/1.4740 |
| | BMT | 1.6491/1.6818/1.6775 |
| | Tween 80 | 1.4415/1.4496/1.4535 |
| | SB3-8 | 1.2558/1.2481/1.2535 |
| | SB3-10 | 1.4640/1.4545/1.4561 |
| | SB3-14 | 1.0397/1.0307/1.0374 |
| | SB3-16 | 1.7098/1.7091/1.7118 |
| Staphylococcus | No addition | 1.4501/1.4531/1.4572 |
| aureus | Tergitol NP-9 | 1.4008/1.4053/1.4054 |
| | Tergitol NP-10 | 1.6421/1.6479/1.6491 |
| | Tergitol NP-40 | 1.4696/1.4681/1.4699 |
| | Triton X-100 | 1.6211/1.6214/1.6183 |
| | BMT | 1.5377/1.5373/1.5397 |
| | Tween 80 | 1.4287/1.4295/1.4301 |
| | SB3-8 | 1.7094/1.7104/1.7109 |
| | SB3-10 | 0.9821/0.9848/0.9928 |
| | SB3-14 | 0.9865/0.9850/0.9808 |
| | SB3-16 | 1.0008/1.0041/1.0063 |

By referring to the table 3 and table 4, the bacterial lysis efficacy of SB3 series appeared to be excellent. A characterized result is that the lysis efficacy appeared to be higher in E. coli O157:H5, Salmonella, and Vibrio, which all are gram-negative bacteria with a thin peptidoglycan layer in the cell wall, whereas the lysis efficacy appeared slightly lower in Listeria and Staphylococcus, which are gram-positive bacteria with a thick peptidoglycan layer in the cell wall.

Among the SB3 series, 3-[Dimethyl (tetradecyl)ammonio]propane-1-sulfonate (SB3-14) showed the best bacterial lysis efficacy.

(2) Bacterial Lysis Efficacy Test for Different Types of Lysis Detergent

Among the SB3 series, 3-[Dimethyl (tetradecyl)ammonio]propane-1-sulfonate (SB3-14) showed the best bacterial lysis efficacy. However, since the result was measured with 1% concentration, among the Triton X-100 and SB3 series that showed relatively better lysis efficacy among the 10 types of detergents, the lysis efficacies of SB3-10, SB3-14, and SB3-16, excluding SB3-8, were tested. The results are listed in the table 5 and table 6 below.

TABLE 5

| Tested Strain | Detergent | 0.1% | 0.5% | 1% | 2% | 3% | 5% |
|---|---|---|---|---|---|---|---|
| Salmonella | Triton X-100 | 1.0968 | 1.0965 | 0.8827 | 0.9457 | 0.8287 | 0.8258 |
| spp. | | 1.0927 | 1.0899 | 0.8879 | 0.9452 | 0.8303 | 0.8240 |
| (initial O.D) = | | 1.0890 | 1.1089 | 0.8963 | 0.9464 | 0.8231 | 0.8249 |
| 1.4448) | SB3-10 | 0.9125 | 0.4241 | 0.4611 | 0.4310 | 0.4184 | 0.9464 |
| | | 0.9111 | 0.4241 | 0.4597 | 0.4310 | 0.4186 | 0.9488 |
| | | 0.9138 | 0.4359 | 0.4705 | 0.4345 | 0.4199 | 0.9479 |
| | SB3-14 | 0.5393 | 0.5807 | 0.4105 | 0.5727 | 0.5196 | 0.4419 |
| | | 0.5423 | 0.5788 | 0.4095 | 0.5754 | 0.5196 | 0.4454 |
| | | 0.5520 | 0.5796 | 0.4095 | 0.5776 | 0.5221 | 0.4443 |
| | SB3-16 | 0.6487 | 0.7598 | 0.4932 | 0.4688 | 0.4487 | 0.4594 |
| | | 0.6399 | 0.7515 | 0.4918 | 0.4680 | 0.4508 | 0.4580 |
| | | 0.6538 | 0.7651 | 0.4892 | 0.4676 | 0.4516 | 0.4632 |
| Vibrio | Triton X-100 | 0.3916 | 0.3421 | 0.2844 | 0.3044 | 0.2947 | 0.2499 |
| vulnificus | | 0.3935 | 0.3417 | 0.2794 | 0.3089 | 0.2950 | 0.2478 |
| (initial O.D = | | 0.3923 | 0.3437 | 0.2801 | 0.3046 | 0.2966 | 0.2431 |
| 0.6143) | SB3-10 | 0.3351 | 0.2124 | 0.1822 | 0.1627 | 0.0869 | 0.1477 |
| | | 0.3199 | 0.2149 | 0.1758 | 0.1647 | 0.0865 | 0.1578 |
| | | 0.3176 | 0.2105 | 0.1891 | 0.1624 | 0.0893 | 0.1677 |
| | SB-14 | 0.1924 | 0.0679 | 0.0601 | 0.0445 | 0.0453 | 0.0310 |
| | | 0.1953 | 0.0739 | 0.0595 | 0.0393 | 0.0547 | 0.0329 |
| | | 0.1984 | 0.0695 | 0.0588 | 0.0447 | 0.0528 | 0.0320 |
| | SB3-16 | 0.1196 | 0.0680 | 0.0553 | 0.0399 | 0.0395 | 0.0324 |
| | | 0.1210 | 0.0627 | 0.0506 | 0.0421 | 0.0407 | 0.0317 |
| | | 0.1170 | 0.0656 | 0.0521 | 0.0383 | 0.0453 | 0.0323 |

TABLE 6

| Tested Strain | Detergent | 0.1% | 0.5% | 1% | 2% | 3% | 5% |
|---|---|---|---|---|---|---|---|
| Esherichia | Triton X-100 | 0.8888 | 0.6047 | 0.9722 | 0.9793 | 0.9052 | 0.9238 |
| coli O157 | | 0.8930 | 0_6002 | 0.9715 | 0.9819 | 0.9049 | 0.9183 |
| (initial O.D = | | 0.8848 | 0.5961 | 0.9720 | 0.9767 | 0.9035 | 0.9254 |
| 0.8139) | SB3-10 | 0.5979 | 0.6803 | 0.5228 | 0.5845 | 0.6355 | 0.8160 |
| | | 0.5907 | 0.6818 | 0.5223 | 0.5861 | 0.6371 | 0.8061 |
| | | 0.5816 | 0.6832 | 0.5253 | 0.5779 | 0.6311 | 0.8134 |
| | SB3-14 | 0.4641 | 0.2262 | 0.0270 | 0.4447 | 0.6228 | 0.6731 |
| | | 0.4483 | 0.2254 | 0.0238 | 0.4452 | 0.6275 | 0.6746 |
| | | 0.4351 | 0.2230 | 0.0244 | 0.4459 | 0.6225 | 0.6733 |
| | SB3-16 | 0.9950 | 1.0314 | 0.8049 | 0.8432 | 0.8710 | 0.9536 |
| | | 1.0011 | 1.0172 | 0.7896 | 0.8338 | 0.8653 | 0.9258 |
| | | 0.9956 | 1.0085 | 0.7856 | 0.8284 | 0.8610 | 0.9244 |
| Listeria | Triton X-100 | 0.7013 | 0.7398 | 0.6622 | 0.7014 | 0.6407 | 0.6213 |
| monocytogenes | | 0.7077 | 0.7403 | 0.6765 | 0.7164 | 0.6609 | 0.6340 |
| (initial O.D = | | 0.7059 | 0.7414 | 0.6766 | 0.7099 | 0.6608 | 0.6310 |
| 0.8336) | SB3-10 | 0.7689 | 0.7694 | 0.7935 | 0.7242 | 0.6917 | 0.8414 |
| | | 0.8003 | 0.7802 | 0.7994 | 0.7341 | 0.7252 | 0.8448 |
| | | 0.7683 | 0.7866 | 0.8055 | 0.7357 | 0.7227 | 0.8431 |
| | SB3-14 | 0.9540 | 0.8154 | 0.5993 | 0.9852 | 0.7006 | 0.7960 |
| | | 0.9653 | 0.8244 | 0.5050 | 0.9975 | 0.6813 | 0.7828 |
| | | 0.9757 | 0.8145 | 0.5950 | 0.9807 | 0.6884 | 0.7815 |
| | SB3-16 | 0.7770 | 0.7764 | 0.8133 | 0.7546 | 0.7213 | 0.6344 |
| | | 0.8027 | 0.7530 | 0.8142 | 0.7725 | 0.7797 | 0.6340 |
| | | 0.8073 | 0.7657 | 0.8238 | 0.7891 | 0.7969 | 0.6042 |
| Staphylococcus | Triton X-100 | 1.1248 | 11919 | 1.2024 | 1.2247 | 1.1855 | 1.2147 |
| aureus | | 1.1255 | 1.1925 | 1.2028 | 1.2245 | 1.1810 | 1.2108 |
| (initial O.D = | | 1.1291 | 1.1898 | 1.2027 | 1.2231 | 1.1826 | 1.2034 |
| 1.1877) | SB3-10 | 0.9335 | 0.9517 | 0.8872 | 0.8495 | 0.5582 | 0.8456 |
| | | 0.9313 | 0.9486 | 0.8856 | 0.8478 | 0.5458 | 0.8446 |
| | | 0.9293 | 0.9454 | 0.8847 | 0.8480 | 0.5385 | 0.8442 |
| | SB3-14 | 0.9052 | 1.1084 | 0.5341 | 0.9473 | 1.0432 | 0.8999 |
| | | 0.9069 | 1.1070 | 0.5322 | 0.9470 | 1.0429 | 0.8987 |
| | | 0.9087 | 1.1092 | 0.532.7 | 0.9461 | 1.0502 | 0.8865 |
| | SB3-16 | 1.0492 | 1.1503 | 1.1763 | 1.0764 | 1.0675 | 1.0721 |
| | | 1.0472 | 11498 | 1.1734 | 1.0741 | 1.0649 | 1.0700 |
| | | 1.0449 | 1.1487 | 1.1719 | 1.0744 | 1.0654 | 1.0711 |

By referring to the table 5 and table 6 above, SB3-14 shows the best bacterial lysis efficacy, and the best bacterial lysis efficacy is expressed at 1% concentration except for Vibrio vulnificus.

(3) Lysis Efficacy Test for Lysis Detergent Additives

For the 1% SB3-14 that showed an excellent bacterial lysis efficacy in the result, efficacies of lysozyme, C7BzO and silica bead (200 mesh) as an additive for enhancing the bacterial lysis efficacy were evaluated. The results are listed in the table 7, table 8 and table 9 below.

TABLE 7

| Detergent | no | +0.1% | +1 mg | +1% |
|---|---|---|---|---|
| (1% SB3-14) | addition | C7BZO | Lysozyme | silica bead |
| Esherichia coli | 0.1112 | 0.0348 | 1.1502 | 0.1820 |
| O157 | 0.1137 | 0.0365 | 1.1501 | 0.1835 |
| (initial O.D = | 0.1126 | 0.0342 | 1.1487 | 0.1807 |
| 1.0911) | | | | |
| Listeria | 0.6514 | 0.5861 | 1.2582 | 0.6945 |
| monocytogenes | 0.6579 | 0.5878 | 1.2597 | 0.6964 |
| (initial O.D = | 0.6484 | 0.5891 | 1.2608 | 0.6966 |
| 1.2010) | | | | |
| Staphylococcus | 0.8154 | 0.7611 | 1.7339 | 0.9316 |
| aureus | 0.8152 | 0.7606 | 1.7341 | 0.9323 |
| (initial O.D = | 0.8153 | 0.7604 | 1.7332 | 0.9319 |
| 1.2274) | | | | |
| Salmonella spp. | 0.3597 | 0.2569 | 1.1859 | 0.3694 |
| (initial O.D = | 0.3519 | 0.2524 | 1.1817 | 0.3699 |
| 1.0894) | 0.3447 | 0.2496 | 1.1746 | 0.3727 |
| Vibrio vulnificus | 0.0800 | 0.0859 | 0.5912 | 0.0989 |
| (initial O.D = | 0.0798 | 0.0860 | 0.5944 | 0.0996 |
| 0.5670) | 0.0787 | 0.0859 | 0.5912 | 0.0990 |

By referring to the table 7, the lysis efficacy appeared to have increased the most when C7BzO and silica bead were added to 1% (v/v) SB3-14.

Meanwhile, the lysis enhancement efficacy for different concentrations of C7BzO addition was investigated. The results are listed in the table 8 below.

TABLE 8

| Detergent | no | +0.1% | +0.2% | +0.3% | 0.5% |
|---|---|---|---|---|---|
| (1% SB3-14) | addition | C7BZO | C7BZO | C7BZO | C7BZO |
| Esherichia coli | 0.1264 | 0.0614 | 0.1798 | 0.1365 | 0.1754 |
| O157 | 0.1254 | 0.0595 | 0.1799 | 0.1371 | 0.1703 |
| (initial O.D = | 0.1241 | 0.0597 | 0.1763 | 0.1344 | 0.1629 |
| 1.0829 ) | | | | | |
| Listeria | 0.7595 | 0.6141 | 0.7108 | 0.7945 | 0.7770 |
| monocytogenes | 0.7673 | 0.6187 | 0.7115 | 0.7949 | 0.7793 |
| (initial O.D = | 0.7609 | 0.6197 | 0.7129 | 0.7953 | 0.7810 |
| 1.1086) | | | | | |
| Staphylococcus | 0.8664 | 0.7039 | 0.9136 | 0.8335 | 0.8793 |
| aureus | 0.8654 | 0.7998 | 0.9103 | 0.8278 | 0.8753 |
| (initial O.D = | 0.8652 | 0.7994 | 0.9109 | 0.8202 | 0.8675 |
| 1.1374) | | | | | |
| Salmonella spp. | 0.3640 | 0.2467 | 0.3636 | 0.3802 | 0.3629 |
| (initial O.D = | 0.3631 | 0.2384 | 0.3636 | 0.3764 | 0.3622 |
| 1.3421) | 0.3627 | 0.2295 | 0.3625 | 0.3841 | 0.3712 |
| Vibrio | 0.2733 | 0.0759 | 0.0734 | 0.0576 | 0.0785 |
| vulnificus | 0.2706 | 0.0863 | 0.0671 | 0.0580 | 0.0813 |
| (initial O.D = | 0.2737 | 0.0854 | 0.0632 | 0.0565 | 0.0808 |
| 1.0272) | | | | | |

By referring to the table 8, the lysis efficacy appeared to have increased the most when 0.1% (v/v) C7BzO was added to 1% (v/v) SB3-14.

Meanwhile, since the lysis level of the gram-positive bacteria are lower than the gram-negative bacteria, the influence of silica bead addition to the bacterial lysis for gram-positive bacteria, Staphylococcus aureus and Listeria monocytogenes, was investigated for more effective bacterial lysis.

TABLE 9

| Detergent | | | | | |
|---|---|---|---|---|---|
| (1% SB3-14 + | no | +0.1% | +0.2% | +0.3% | +0.4% |
| 5% silica bead) | addition | C7BZO | C7BZO | C7BZO | C7BZO |
| Listeria | 0.7109 | 0.5856 | 0.6074 | 0.6158 | 0.6733 |
| monocytogenes | 0.7265 | 0.5779 | 0.6092 | 0.6117 | 0.6717 |
| (initial O.D = | 0.7268 | 0.5823 | 0.6161 | 0.6190 | 0.6734 |
| 1.1391) | | | | | |
| Staphylococcus | 1.1501 | 0.9484 | 0.9961 | 1.0868 | 1.1901 |
| aureus | 1.1538 | 0.9487 | 0.9964 | 1.0846 | 1.2547 |
| (initial O.D = | 1.1502 | 0.9491 | 0.9975 | 1.0866 | 1.2543 |
| 1.4663) | | | | | |

By referring to the table 9, the addition of silica bead was identified to bring a significant synergy effect to the bacterial lysis for gram-positive bacteria, Staphylococcus aureus and Listeria monocytogenes.

(4) Lysis Efficacy Test of Lysis Reagent Composition

(A) Test for Identifying Bacterial Lysis Efficacy of Lysis Reagent Composition Using SDS-PAGE

According to the result, to identify the bacterial lysis efficacy for a composition in which 1% SB3-14 and 0.1% C7BzO are added using phosphate saline buffer (PSB) as the base buffer, the terminal lysis composition for 5 food risk microorganisms, each microorganism was cultured for 24 hours, cells were collected by precipitating with a centrifuge, 0.5 mL lysis reagent was added, centrifuge was done again, protein electrophoresis was performed for 20 mL of the each supernatant, and the bacterial lysis efficacy was identified with SDS-PAGE.

Meanwhile, to identify the bacterial lysis efficacy of a lysis reagent composition for 5 types of food risk microorganisms, a comparative test was performed by purchasing and using the lysis buffer that was commonly used before (50 mM Tris pH 8.0, 0.1% Triton X-100, 0.1 mg lysozyme) and a commercially used product of Thermo Co., B-PER buffer. Here, a mutual comparison was made for cases in which silica bead was added and was not added.

The result is shown in FIG. 1.

As shown in FIG. 1, lysis did not occur in food risk microorganisms except Vibrio vulnificus when a simple phosphate buffer solution was used. The developed lysis reagent composition was identified to have extracted more protein, than the lysis buffer that was commonly used before and commercial B-PER buffer. Specifically, the bacterial lysis efficacy was noticeably higher in the gram-positive bacteria than the gram-negative bacteria, and as a result of comparing the case of using only the lysis reagent of the present invention and the case of adding silica bead thereto, the case of addition was identified to have better lysis efficacy.

(B) Test for Identifying Bacterial Lysis Efficacy of Lysis Reagent Composition Using Bicinchoninic Acid (BCA) Assay

Further, according to the result, to identify the bacterial lysis efficacy of (i) a composition in which 1% SB3-14 and 0.1% C7BzO are added using phosphate saline buffer (PSB) as the base buffer; or a composition in which 1% SB3-14, 0.1% C7BzO and 1% silica bead are added using phosphate saline buffer (PSB) as the base buffer, 5 food risk microorganisms were cultured for 24 hours, cells were collected by precipitating them with a centrifuge, 0.5 mL of the lysis reagent composition was added, centrifuge was done again, and supernatant was collected.

The bacterial lysis efficacy for 5 food risk microorganisms by the lysis reagent composition was identified by analyzing the total content of the protein included in the supernatant with BCA assay.

For the control group, a normal lysis buffer and a commercial product (B-per) were used. For the normal lysis buffer, 0.1% Trioton X-100 and 100 mg of Lysozyme were added using 50 mM Tri-HCl (pH 8.0) as the base buffer, and B-PER™ Bacterial Protein Extraction Reagent produced by Thermo fischer Co. was used for the commercial product.

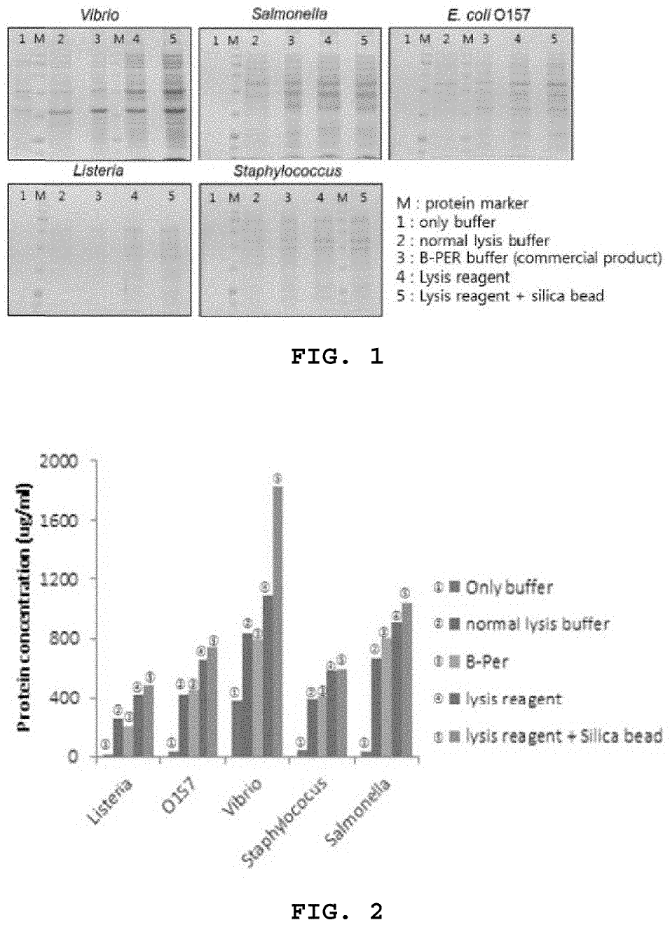

The result is shown in FIG. 2.

As shown in FIG. 2, as the result of comparing the bacterial lysis efficacy for each condition, the lysis reagent composition developed by the Example 1 of the present invention was found to show more effective bacterial lysis compared to the previously used normal lysis buffer or the commercialized product, and adding silica bead was found to have better lysis efficacy.

Through the results, terminal decision was made to use phosphate saline buffer (PSB) as the base buffer and comprise 1% (v/v) SB3-14 and 0.1% (v/v) C7BzO for the lysis reagent composition to be applied to 5 types of food risk microorganisms, and decision was made to add silica bead to provide a higher synergy effect.

### Example 3

1. Selection of Chromogenic Reagent for Detecting a Microorganism

9 chromogenic reagents were purchased and utilized to select a chromogenic reagent for a microorganism. The list is shown in the table 10 below.

TABLE 10

| o. | Chromogenic substrates | Cas No. | Reaction color |
|---|---|---|---|
| | Aldol ® 518 myo-inositol-1-phosphate | (P) US | Red |
| | | 8940909 | |
| | 5-Bromo-4-chloro-3-indoxyl phosphate, disodium salt | 102185-33-1 | Blue |
| | (X-phos • 2Na) | | |
| | 6-Chloro-3-indoxyl-beta-D-glucopyranoside | 159954-28-6 | Purple |
| | (Salmon-α-D-Glc) | | |
| | 6-Chloro-3-indoxyl-alpha-D-glucopyranoside | 467214-46-6 | Purple |
| | (Salmon-α-D-Glc) | | |
| | 5-Bromo-4-chloro-3-indoxyl-beta-D-glucuronic acid | 370100-64-4 | Blue |
| | Sodium salt trihydrate (X-beta-D-GlcA • 2Na) | | |
| | 5-Bromo-6-chloro-3-indoxyl-beta-D-galactopyranoside | 93863-88-8 | Purple |
| | (Magenta-beta-D-Gal) | | |
| | 5-Bromo-6-chloro-3-indoxyl caprylate | 209347-94-4 | Blue |
| | (Magenta-caprylate) | | |
| | 5-Bromo-4-chloro-3-indoxyl-beta-D-glucopyranoside | 15548-60-4 | Blue |
| | (X-beta-D-Glc) | | |
| | 5-Bromo-4-chloro-3-indoxyl-alpha-D-galactopyranoside | 107021-38-5 | Purple |
| | (X-alpha-Gal) | | |

Since the chromogenic reagents have low water solubility, every chromogenic reagent except X-phosphate was dissolved in dimethyl sulfoxide (DMSO) for utilization and X-phosphate was dissolved in tertiary distilled water.

To detect coloring reaction of chromogenic reagent to 5 types of food risk microorganisms, the chromogenic reagents were dissolved to have 100 mM concentration and were used as the stock solution, and were added to have the terminal concentration of 10 mM and coloring reaction was tested.

The 5 types of food risk microorganisms were used for the coloring reaction test by inoculating the strains pre-cultured for 24 hours at inoculum volume of 1%, culturing for 24 hours in the culture medium, and regulating the number of bacteria to be similar to the aforementioned number of bacteria by measuring the OD.

Specifically, for the coloring reaction test for each microorganism, 1.5 mL of each microorganism culture solution cultured in said conditions were centrifuged, bacterial cells were collected, suspensions were prepared by adding 0.5 mL of lysis reagent composition prepared from the Example 2, and disruption reaction was performed for 5-10 minutes with a sonicator or a vortex mixer.

After the disruption reaction, centrifuge was performed again, 0.1 mL of each supernatant was added on a 96 well plate, 10 mL of pre-prepared 100 mM stock solution for each chromogenic reagent was added, reaction was performed for 30 minutes at 37° C., and the matter of coloring reaction was detected.

The result is shown in FIG. 3-9.

As shown in FIG. 3, Vibrio vulnificus showed unique coloring reaction to each of Magenta-beta-galactopyranoside, Salmon-alpha-glucospyranoside, Magenta-beta-glucopyranoside and X-alpha-glucospyranoside under the aforementioned condition. Among the reagents, Magenta-beta-galactopyranoside (purple) and X-alpha-glucospyranoside (blue) were selected as chromogenic reagents for detecting the Vibrio vulnificus.

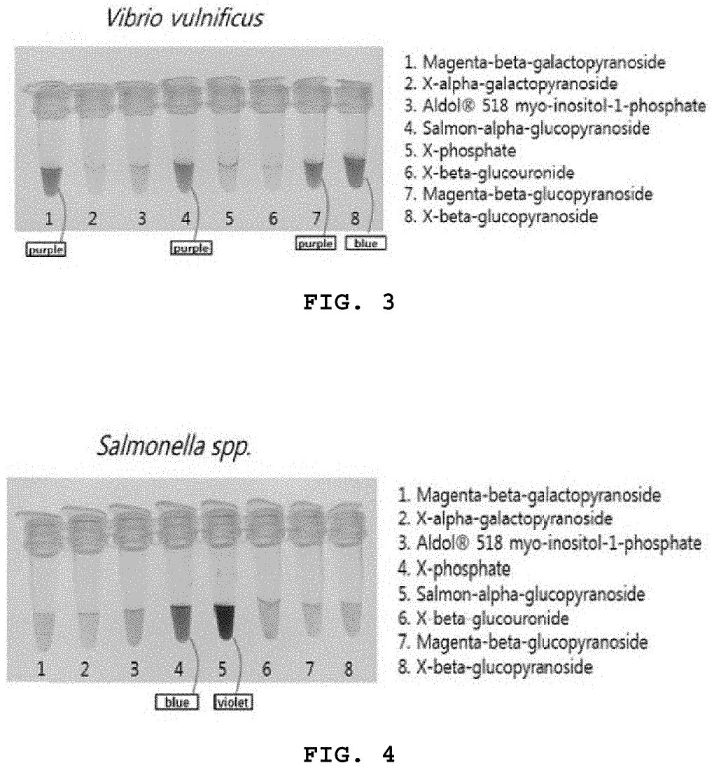

As shown in FIG. 4, Salmonella spp. showed unique and strong coloring reaction to each of X-phosphate and Salmon-alpha-glucospyranoside under the aforementioned condition. Among the two substrates that showed strong coloring reaction, X-phosphate (blue) and Salmon-alpha-glucospyranoside (purple) were selected as chromogenic reagents for detecting the Salmonella spp.

As shown in FIG. 5, as a result of testing lipase activity response for selective detection of food risk microorganisms, other food risk microorganisms did not show lipase activity response, and although Staphylococcus aureus and Salmonella spp. showed coloring reaction, the Salmonella spp. showed higher intensity and speed in the reaction and hence Magenta-caprylate (purple) was selected as the chromogenic substrate for detecting the Salmonella spp.

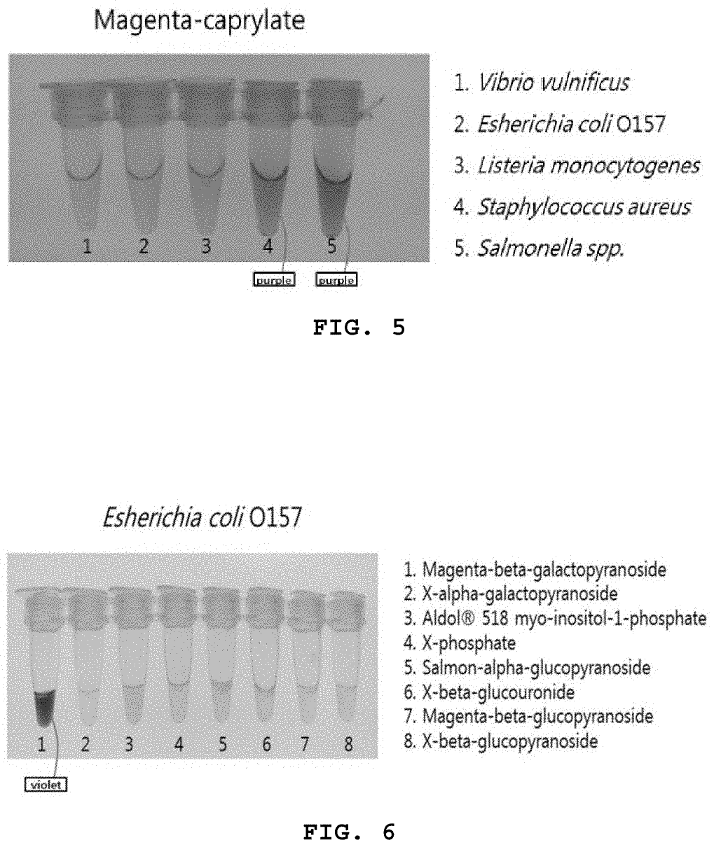

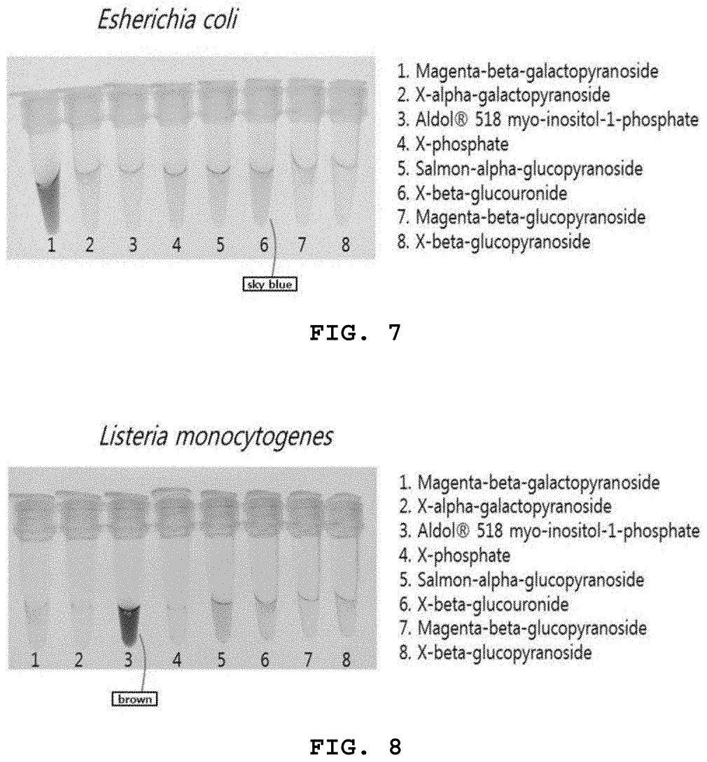

As shown in FIG. 6, Escherichia coli O157 showed unique coloring reaction only for Magenta-beta-galactopyranoside (purple) under the aforementioned condition.

As shown in FIG. 7, Escherichia coli showed unique coloring reaction for X-beta-glucouronide (light blue) under the aforementioned condition.

As shown in FIG. 8, Listeria monocytogenes showed unique coloring reaction for Aldol-myo-inositol-1-phosphate (brown) under the aforementioned condition.

As shown in FIG. 9, Staphylococcus aureus showed each unique coloring reaction for Magenta-beta-galactopyranoside, X-phosphate and Salmon-alpha-glucospyranoside under the aforementioned condition. Among the reagents, Salmon-alpha-glucospyranoside (purple) and X-phosphate (blue) were selected as chromogenic reagents for detecting the Staphylococcus aureus.

2. Coloring Reaction Test for Different Concentrations of Chromogenic Reagent

For the coloring reaction test for different concentrations of the selected chromogenic reagents, 1.5 mL of food risk microorganism cultured in said conditions were centrifuged, bacterial cells were collected, suspensions were prepared by adding 0.5 mL of lysis reagent composition, and disruption reaction was performed for 5-10 minutes with a sonicator or a vortex mixer. After the disruption reaction, centrifuge was performed again, 0.1 mL of each supernatant was added on a 96 well plate, 10 μL of pre-prepared 100, 50, 40, 30, 20, 10, 5, 1 mM stock solutions for each chromogenic reagent were added, reaction was performed for 30 minutes at 37° C., and the matter of coloring reaction was detected as follows.

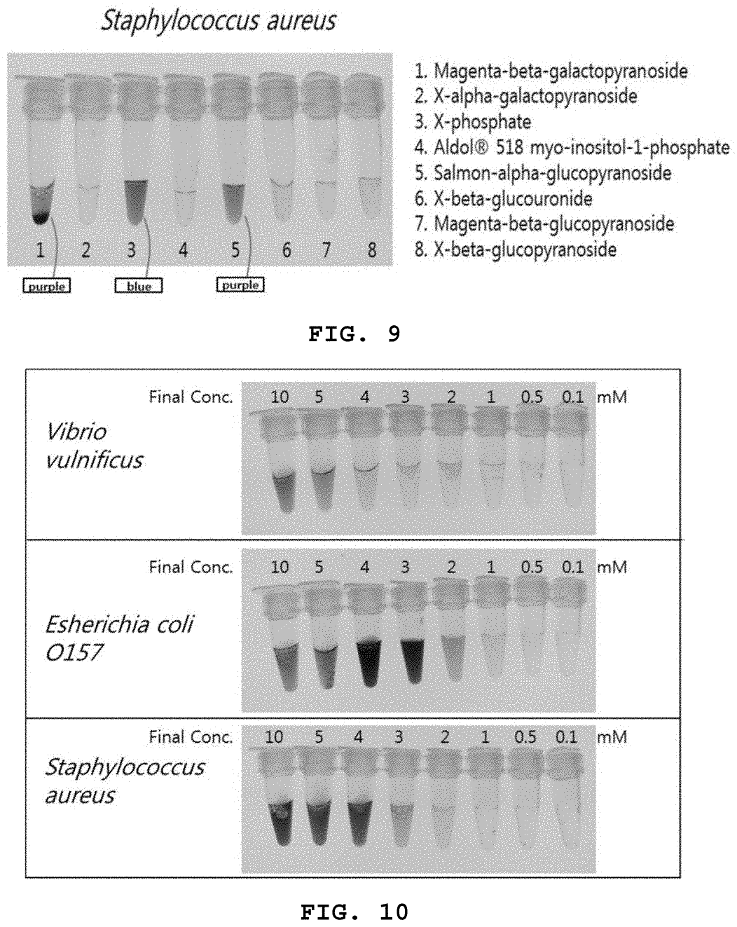

The result is shown in FIG. 10-15.

As shown in FIG. 10, Vibrio vulnificus showed a strong coloring reaction when the concentration of Magenta-beta-galactopyranoside was 100 mM and the final concentration was 5-10 mM, and preferably showed the strongest coloring reaction at 10 mM. Staphylococcus aureus showed a strong coloring reaction when the concentration of Magenta-beta-galactopyranoside was 100 mM and the final concentration was 4-10 mM, and preferably showed the strongest coloring reaction at 10 mM. Escherichia coli O157 showed a strong coloring reaction when the concentration was 40 mM and the final concentration was 3-10 mM, and preferably showed the strongest coloring reaction at 3-4 mM.

As shown in FIG. 11, a strong coloring reaction occurred when the concentration of X-beta-glucopyranoside was 100 mM and the final concentration was 5-10 mM, and preferably the coloring reaction was the strongest at 10 mM.

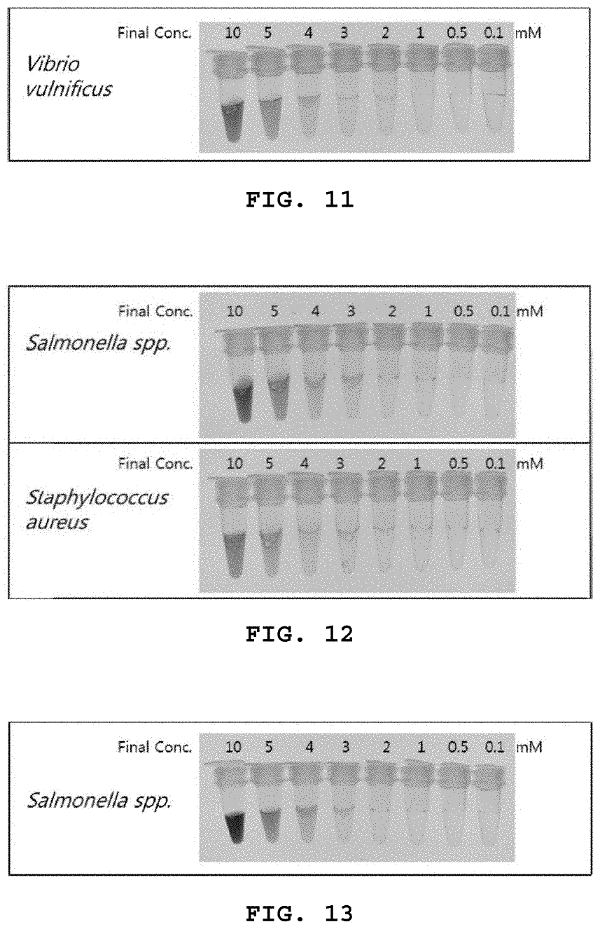

As shown in FIG. 12, both Salmonella spp. and Staphylococcus aureus showed strong coloring reactions when the concentration of X-phosphate was 100 mM and the final concentration was 5-10 mM, and preferably showed the strongest coloring reaction at 10 mM.

As shown in FIG. 13, a strong coloring reaction occurred when the concentration of Magenta-caprylate was 100 mM and the final concentration was 5-10 mM, and preferably the coloring reaction was the strongest at 10 mM.

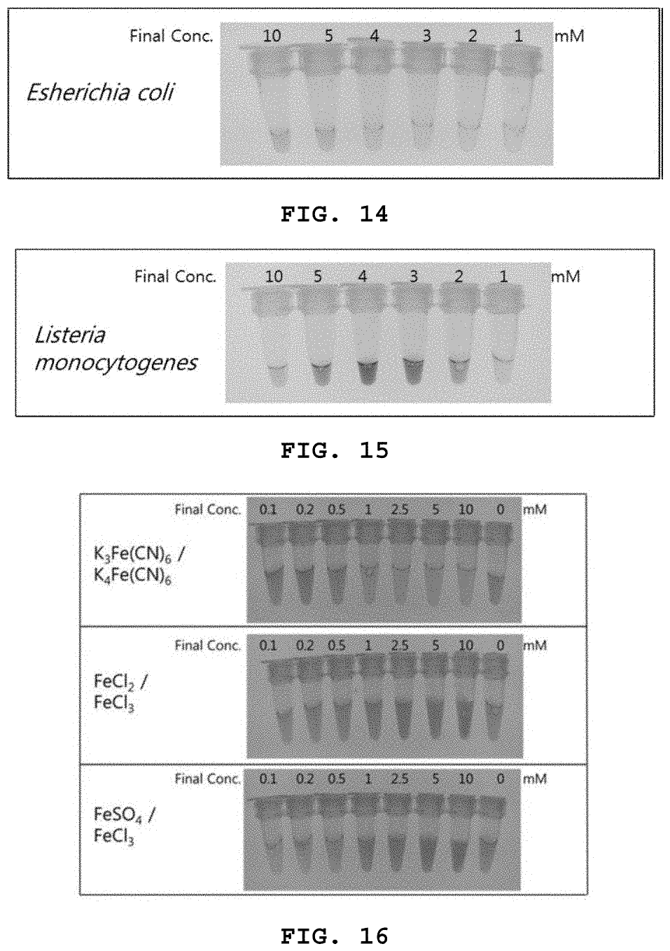

As shown in FIG. 14, a strong coloring reaction occurred when the concentration of X-beta-glucuronide was 100 mM and the final concentration was 5-10 mM, and preferably the coloring reaction was the strongest at 10 mM.

As shown in FIG. 15, a strong coloring reaction occurred when the concentration of Aldol-myo-inositol-1-phosphate was 40 mM and the final concentration was 2-4 mM, and preferably the coloring reaction was the strongest at 4 mM.

### Example 4

Selection of Oxidation Reagent for Detecting a Microorganism

An oxidation reagent was to be developed to promote oxidation of chromophore in a coloring reaction process of a chromogenic reagent when detecting a microorganism. To this end, 1.5 mL of each microorganism culture solution cultured in said conditions were centrifuged, bacterial cells were collected, suspensions were prepared by adding 0.5 mL of lysis reagent composition prepared from the Example 2, and disruption reaction was performed for each reaction time with a sonicator.

After the disruption reaction, centrifuge was performed again, 0.1 mL of each supernatant was added on a 96 well plate, 10 μL of each pre-prepared chromogenic reagent was added, potassium ferriccyanide (K₃Fe(CN)₆) and potassium ferrocyanide (K₄Fe(CN)₆); FeCl₂ and FeCl₃; and FeSO₄ and FeCl₂ were added at each concentration as oxidation reagents, reaction was performed for 30 minutes at 37° C., and the matter of coloring reaction was tested. As for the chromogenic reagent, Magenta-beta-galactopyranoside was used for Vibrio vulnificus, Magenta-caprylate was used for Salmonella spp., X-phosphate was used for Staphylococcus aureus, Aldo-myo-inositol phosphate was used for Listeria monocytogenes, and Magenta-beta-galactopyranoside was used for Escherichia coli O157.

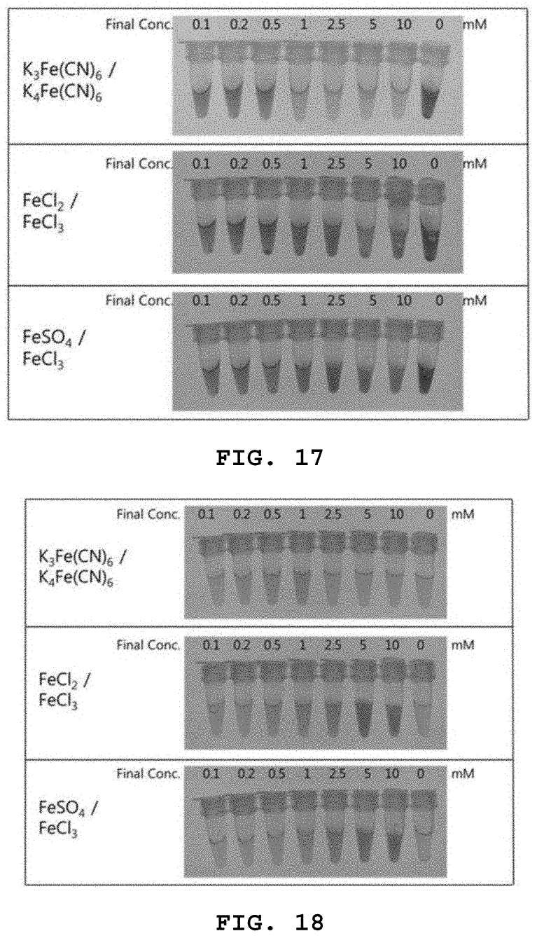

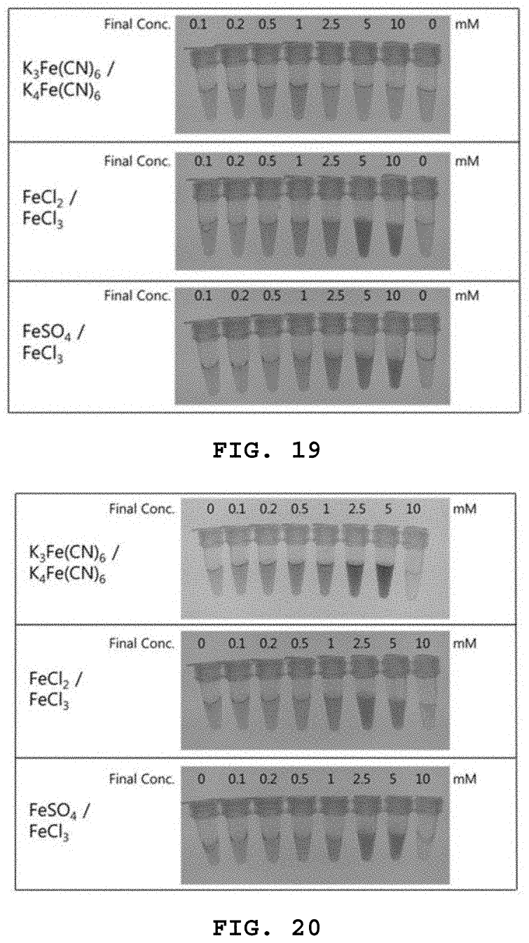

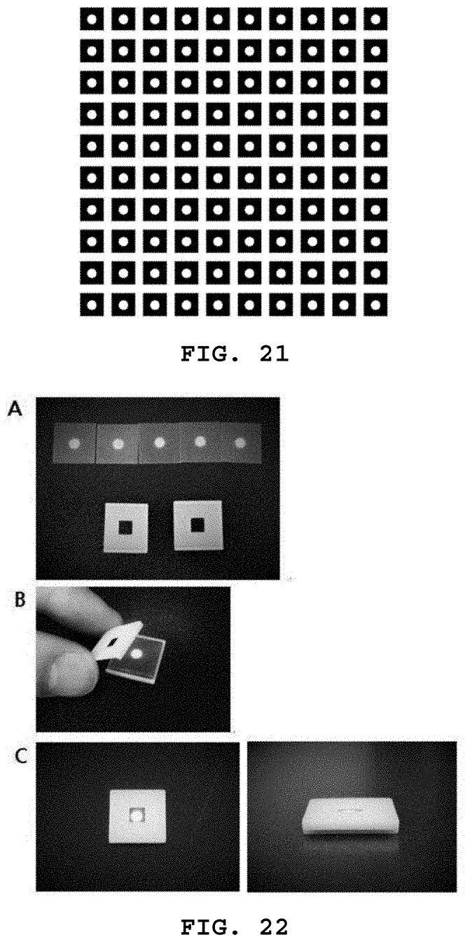

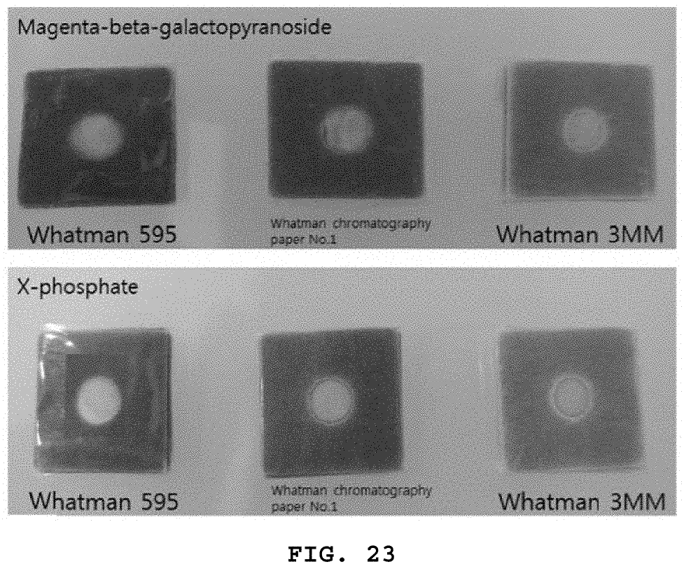

The results are shown in FIG. 16-20.

As shown in FIG. 16-20, there were results in which adding an oxidation reagent promoted coloring reaction more than not adding the oxidation reagent, but most of the obtained results showed that adding an oxidation reagent did not influence the coloring reaction or even reduced the coloring reaction.

For Magenta-beta-glucopyranoside, Magenta-beta-galactopyranoside, or X-phosphate, adding potassium ferriccyanide (K₃Fe(CN)₆) and potassium ferrocyanide (K₄Fe(CN)₆) particularly showed to promote coloring reaction. Here, the best coloring reaction results were obtained when the final concentrations were 0.2, 2.5 and 0.5 mM, respectively.

For potassium ferriccyanide (K₃Fe(CN)₆)/potassium ferrocyanide (K₄Fe(CN)₆), FeCl₂/FeCl₃, and FeSO₄/FeCl₃, the results were either similar with potassium ferriccyanide (K₃Fe(CN)₆)/potassium ferrocyanide (K₄Fe(CN)₆) or were even found to lower the coloring reactions, so potassium ferriccyanide (K₃Fe(CN)₆)/potassium ferrocyanide (K₄Fe(CN)₆) were considered to be appropriate for oxidative reagents.

However, in case of detecting Listeria monocytogenes using Aldol-myo-inositol-phosphate as a coloring reagent, potassium ferriccyanide (K₃Fe(CN))₆/potassium ferrocyanide (K₄Fe(CN)₆) showed a result of lowering the coloring reaction. Here, the added potassium ferriccyanide (K₃Fe(CN)₆)/potassium ferrocyanide (K₄Fe(CN)₆) seems to rapidly inhibit the activity of an enzyme that creates the coloring reaction. Therefore FeCl₂/FeCl₃ and FeSO₄/FeCl₃ were considered to be appropriate for oxidation reagents when detecting Listeria monocytogenes.

### Example 5

Preparation of a Microfluidic Paper Chip Using a Solid Wax Printing Technology

(1) Preparation of a Wax-Printed Paper Media

Chromatography paper No. 1, chromatography paper 3 MM, filter paper Grade 4, and filter paper No. 595 of Whatman Co., and filter paper No. 100 and No. 22 of Hyundai Micro Co. were the paper media used for raw material of the microfluidic paper chip. Colorqube 8870 of Xerox Co. was used as a printer to print wax, and HP330D of Misung Co. was used as a heater. The thickness and pore sizes of each paper media are listed in the Table 11 below.

TABLE 11

| | Thickness | pore size |
|---|---|---|
| Paper | (μm) | (μm) |
| Whatman Chromatography 3MM | 340 | 12 |
| Whatman chromatography No. 1 | 180 | 12 |
| Whatman filter grade 595 | 160 | 7 |
| Whatman filter grade 4 | 205 | 23 |
| Hyundai No. 22 | 210 | 14 |
| Hyundai No. 100 | 160 | 3 |

Clewin 3, an economic layout design program, was used for producing a design. The design was produced by overlapping the hydrophobic layer and the hydrophilic layer of the paper microfluidic device and removing the overlapped area of the hydrophobic layer.

When printing the produced design on a paper medium, the size of the printing paper was set to be 200×200 (mm). To place sufficient amount of solid wax, the printing quality was set to be ‘Photo’.

The printed paper was heated for a certain amount of time in the heater. To prevent any contamination by wax and other substances remaining in the heater, a sweeper or an aluminum foil was used. To apply heat equally on the whole paper, an object with some weight was placed on the aluminum foil.

The design of the paper medium prepared by the method is shown in FIG. 21.

When referring to FIG. 21, the areas shown in black in each small square are coated with wax and are hydrophobic, and the white circle areas are the paper medium, showing the hydrophilic areas.

(2) Preparation of a Microfluidic Paper Chip

The wax-printed paper medium prepared by the method was sliced into small squares and used for preparing a microfluidic paper chip. The microfluidic paper chip was prepared by laminating the sliced paper medium into total of 5 layers.

Each layer was prepared to show the conformations and functions as follows.

The first layer is an inlet layer in which a sample to be detected is injected, and the paper medium is intactly used without any treatment.

The second layer is a lysis layer that lyses a microorganism existing in the sample, and is prepared by absorbing the lysis reagent composition prepared from the example 2 to the hydrophilic area of the paper medium and drying.

The third layer is an oxidation layer in which an oxidation reagent is added to promote oxidation of a chromophore in a coloring reaction of a chromogenic reagent during a detection of a microorganism, and is prepared by absorbing the oxidation reagent prepared from the example 4 to the hydrophilic area of the paper medium and drying.

The fourth layer is a chromogenic layer that forms colors to enable a unique coloring reaction to appear when a microorganism to be detected exists in a sample, and is prepared by absorbing each of the chromogenic reagents prepared from the example 3 to the hydrophilic area of the paper medium and drying.

The fifth layer is an outer layer where the detection result by the coloring reaction appears, from which the existence of the microorganism to be detected by the experimenter can be visually identified, and the paper medium is intactly used without any treatment.

After preparing the paper media from the first layer to the fifth layer, the microfluidic paper chip in the final form was prepared by laminating the layers sequentially and attaching the laminated paper media on a caster that has a hole formed on the top to which a sample can be injected and a hole formed on the bottom from which the coloring result can be observed.

The assembly steps and the final completed form of the microfluidic paper chip are shown in FIG. 22.

(3) Evaluation of Coloring Reaction for Different Thicknesses of Paper Media

To evaluate the effect on coloring reaction by different thicknesses of paper media used for preparing a microfluidic paper chip, a paper medium was prepared according to said (1) method using Whatman filter grade 595 (160 μm thickness), Whatman chromatography paper No. 1 (180 μm) and Whatman chromatography 3 mm (340 μm). Here, the radius of the hydrophilic area with no wax coating was made to be 3 mm.

Then, a microfluidic paper chip was prepared according to said (2) method. Specifically,

(i) Each said paper media was prepared for the first layer.

(ii) The second layer was prepared by thoroughly absorbing a lysis reagent composition that uses phosphate saline buffer (PSB) as the base buffer and comprises 1% (v/v) SB3-14 and 0.1% (v/v) C7BzO to the hydrophilic areas of each said paper media and drying.

(iii) The third layer was prepared by thoroughly absorbing a 10 mM oxidation reagent (K₃Fe(CN)₆)/(K₄Fe(CN)₆) to the hydrophilic areas of each said paper media and drying.

(iv) The fourth layer was prepared by thoroughly absorbing a 50 mM chromogenic reagent, Magenta-beta-galactopyranoside or X-phosphate, to the hydrophilic areas of each said paper media and drying.

(v) Each said paper media with wax coating was prepared for the first layer.

After preparing a microfluidic paper chip by sequentially laminating the paper media of the first to the fifth layer prepared according to said methods, 50 μL of Escherichia coli O157 culture medium was injected through the first layer to the paper chip that used Magenta-beta-galactopyranoside as the chromogenic reagent, 50 μL of Staphylococcus aureus culture medium was injected through the first layer to the paper chip that used X-phosphate as the chromogenic reagent, and reaction was performed for 30 minutes at 37° C.

The result is shown in FIG. 23.

As shown in FIG. 23, every expected coloring reaction was observed regardless of the thickness of the paper media. However, the degree of coloring reaction was found to be more stable as the thickness of the paper medium was thicker. The reason thereof is considered to be that as the thickness of paper increases, a reaction space in which each bacterial lysis reaction, oxidation reaction, and coloring reaction can occur can be stably provided to a certain extent. Therefore, Whatman 3 mm (340 μm), the thickest medium, was selected for utilization.

(4) Evaluation of Coloring Reaction for Different Pore Sizes of Paper Media

To evaluate the effect on coloring reaction by different pore sizes of paper media used for preparing a microfluidic paper chip, Hyundai No. 100 (3 μm), Hyundai No. 22 (14 μm), and Whatman filter grade No. 4 (23 μm) were used. The detailed experiment methods were performed identically with said (3).

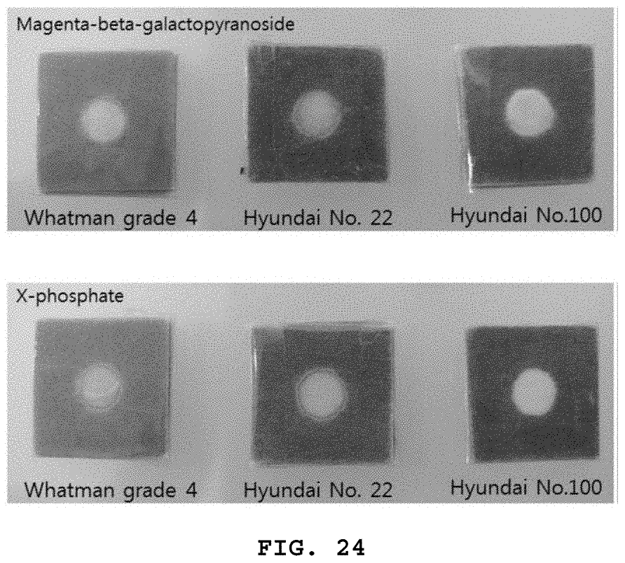

The result is shown in FIG. 24.

As shown in FIG. 24, the coloring reaction did not take place properly when the pore size was too small (Hyundai No. 100) and the coloring reaction occurred appropriately in all other cases. Thus, the appropriate pore size was considered to be 7-23 μm. Thus, Whatman 3 MM (thickness: 340 μm/pore size: 12 μm) having an appropriate thickness and pore size was selected as a major paper material and microfluidic paper chips were to be prepared using same. Here, the paper at the bottom detection area is an area for final confirmation of the reaction, thus Whatman filter grade 4 (thickness: 205 μm/pore size: 3 μm), having the largest pore size among the paper materials, was selected for utilization.

(5) Evaluation of Coloring Reaction for Different Hydrophilic Area Diameters of Paper Media

The degree of coloring reaction for different hydrophilic paper area sizes of the paper media in preparing microfluidic paper chips for detecting a microorganism was to be evaluated.

Whatman chromatography 3 MM (thickness: 340 μm/pore size: 12 μm) was selected as a major paper material, the paper media were wax-coated to have 4, 6, or 8 mm diameter of hydrophilic area, and the coloring reaction was observed with a method identical to said method of (3).

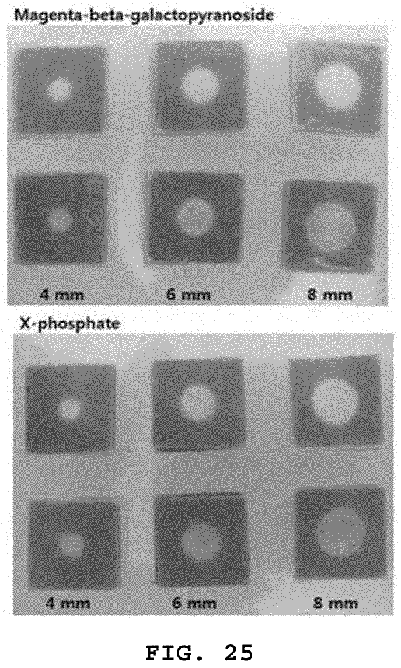

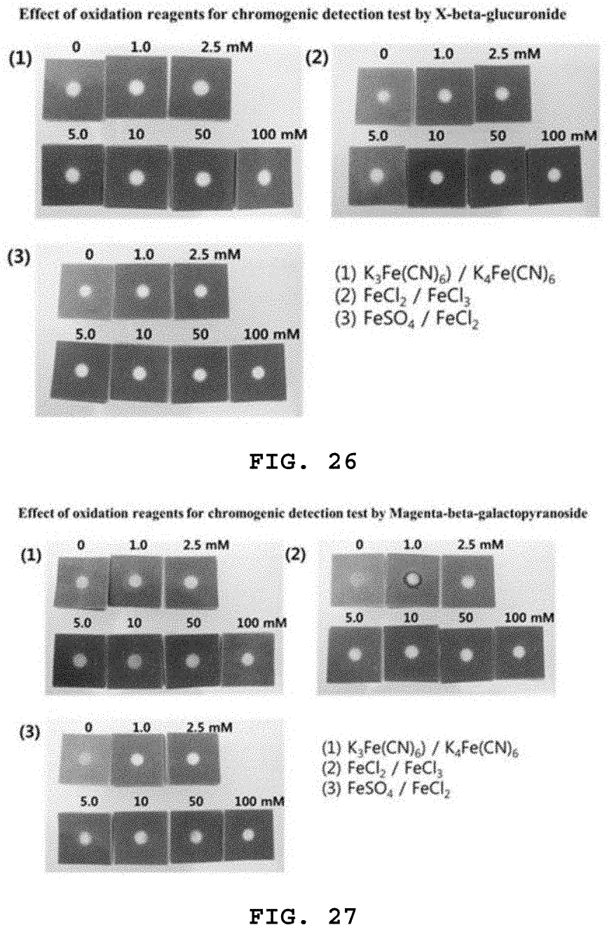

The result is shown in FIG. 25.

As shown in FIG. 25, appropriate coloring reactions occurred regardless of the sizes of the hydrophilic areas. The quantity of reagents needed is different for each sizes of the hydrophilic areas, where each 3 μL of lysis reagent, oxidation reagent, and chromogenic reagent was needed for 4 mm, 5 μL for 6 mm, and 10 μL for 8 mm. Further, the quantity of samples needed is different for each sizes of the hydrophilic areas, where 20, 50, and 100 μL of samples were needed, respectively.

In accordance with such, the 6 mm size of hydrophilic area paper patterns was selected as an appropriate paper pattern of the hydrophilic area, since for the quantity of the needed reagent, particularly the chromogenic reagent is more expensive than other reagents, it is advantageous to use a smaller quantity of reagent in developing an economical paper-based microfluidic device and the size of the hydrophilic area requires an appropriate quantity of sample, thus the diameter of the microfluidic paper chip for a single detection was selected to be 6 mm.

### Example 6

Evaluation on Detection of Escherichia coli O157 and Escherichia coli Using a Microfluidic Paper Chip

(A) Coloring Test of Microfluidic Paper Chip for Different Types and Concentrations of Oxidation Reagents

An investigation for an appropriate type and concentration of oxidation reagent was carried out according to the example 4 to prepare a microfluidic paper chip for detecting Escherichia coli O157.

An investigation for a composition for developing an oxidation reagent was carried out to promote the oxidation of chromophore during a coloring reaction of the chromogenic reagent in detecting Escherichia coli O157 or Escherichia coli. To this end, 1.5 mL of Escherichia coli O157 or Escherichia coli cultured in said conditions was centrifuged, bacterial cells were collected, suspension was prepared by adding 0.5 mL of PBS, and was used as the sample.

Each 5 μL of potassium ferriccyanide (K₃Fe(CN)₆) and potassium ferrocyanide (K₄Fe(CN)₆) was loaded as oxidation reagents on papers that were prepared with pre-prepared patterns for different concentrations of FeCl₂ and FeCl₃, and FeSO₄ and FeCl₂, and was dried for 30 minutes in a 40° C. dryer.

Other than the oxidation reagents, each 5 μL of the lysis reagents prepared in said conditions that are necessary for the microfluidic paper chip assembly and the corresponding chromogenic reagents was loaded as oxidation reagents on papers that were prepared with pre-prepared patterns, and was dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), each 50 μL of pre-prepared Escherichia coli O157 or Escherichia coli suspension was injected thereto, reaction was carried out for 30 minutes at 37° C., and coloring reactions for different types and concentrations of the oxidation reagents were tested.

The results are shown in FIGS. 26 and 27.

As shown in FIGS. 26 and 27, characteristics of oxidation reactions for the two chromogenic reagents used for detecting E. coli: O157, Magenta-beta-galactopyranoside and X-beta-glucuronide, were identified. Magenta-beta-galactopyranoside showed the best coloring reaction with 10 mM potassium ferriccyanide (K₃Fe(CN)₆) and potassium ferrocyanide (K₄Fe(CN)₆).

The oxidation reaction for X-beta-glucuronide did not carry out an oxidation-promoting reaction by an oxidation reagent, and was rather found to inhibit the coloring reaction at concentrations of 50 mM or higher.

(B) Coloring Test of Microfluidic Paper Chip for Different Types and Concentrations of Chromogenic Reagents

An investigation for an appropriate type and concentration of chromogenic reagent was carried out according to the example 3 to prepare a microfluidic paper chip for detecting Escherichia coli O157.

An investigation for an optimized concentration of chromogenic reagent in coloring detection was carried out using Magenta-beta-galactopyranoside as the chromogenic reagent in detecting Escherichia coli O157. Additionally, an investigation for an optimized concentration of chromogenic reagent in coloring detection of X-beta-glucuronide that is used to distinguish the coloring detection of Escherichia coli O157 was carried out for Escherichia coli. To this end, 1.5 mL of Escherichia coli O157 or Escherichia coli cultured in said conditions was centrifuged, bacterial cells were collected, suspension was prepared by adding 0.5 mL of PBS, and was used as the sample.

To investigate the optimized concentration of a chromogenic reagent for detecting Escherichia coli O157, each 5 μL of 5, 10, 25, 50, 100, and 200 mM chromogenic reagents was loaded on papers that were prepared with pre-prepared patterns, and was dried for 30 minutes in a 40° C. dryer.

For the oxidation reagent used in preparing a microfluidic paper chip for detecting Escherichia coli O157, each 5 UL of 10 mM potassium ferriccyanide (K₃Fe(CN)₆) and potassium ferrocyanide (K₄Fe(CN)₆) were loaded as oxidation reagents on papers that were prepared with pre-prepared patterns and were dried for 30 minutes in a 40° C. dryer.

Furthermore, each 5 μL of the lysis reagents developed in said conditions needed for microfluidic paper chip assembly was loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), each 50 μL of pre-prepared Escherichia coli O157 or Escherichia coli suspension was injected thereto, reaction was carried out for 30 minutes at 37° C., and coloring reactions for different types (Magenta-beta-galactopyranoside and X-beta-glucuronide) and concentrations of the chromogenic reagents were tested.

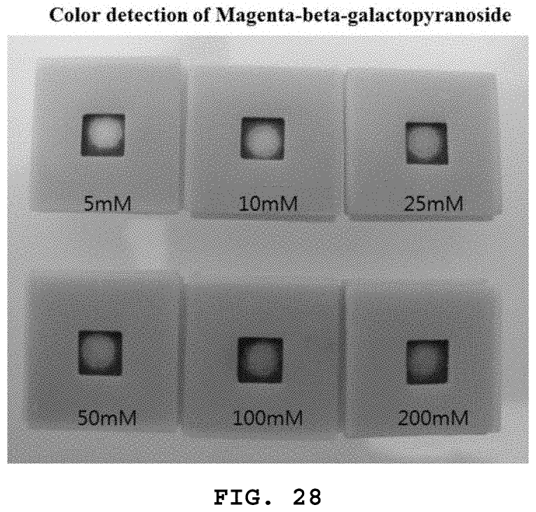

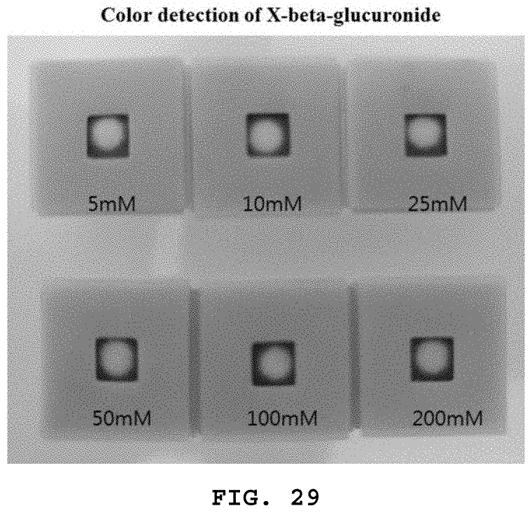

The results are shown in FIGS. 28 and 29.

As shown in FIG. 28, characteristics of coloring reactions for different concentrations of Magenta-beta-galactopyranoside in detecting E. coli: O157 were identified. It was identified that as the concentration of Magenta-beta-galactopyranoside increased, the intensity of the coloring reaction increased. Thus, the concentration of Magenta-beta-galactopyranoside is preferably 25-200 mM, and most preferably 100 mM.

Furthermore, as shown in FIG. 29, as a result of investigating the coloring reaction for different concentrations of X-beta-glucuronide in detecting E. coli, the degree of the coloring reaction increased as the concentration of X-beta-glucuronide increased, and the degree of the coloring reaction rather decreased at 200 mM concentration. Thus, the concentration of X-beta-glucuronide is preferably 25-200 mM, and most preferably 100 mM.

(C) Coloring Test of Microfluidic Paper Chip for Mixed Concentrations of Magenta-Beta-Galactopyranoside and X-Beta-Glucuronide

Meanwhile, by referring to said results, the concentration mixture ratio of the two chromogenic reagents was investigated for an appropriate detection of Escherichia coli O157. To this end, each 5 μL of 100 mM X-beta-glucuronide was loaded on a paper that was prepared with pre-prepared patterns and was dried for 30 minutes in a 40° C. dryer. Afterwards 5 μL of Magenta-beta-galactopyranoside was mixed at different concentrations on the same paper and loaded on the paper that was prepared with pre-prepared patterns and was again dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), each 50 μL of pre-prepared Escherichia coli O157 or Escherichia coli suspension was injected thereto, reaction was carried out for 30 minutes at 37° C., and coloring reactions for the mixture of the two chromogenic reagents were tested.

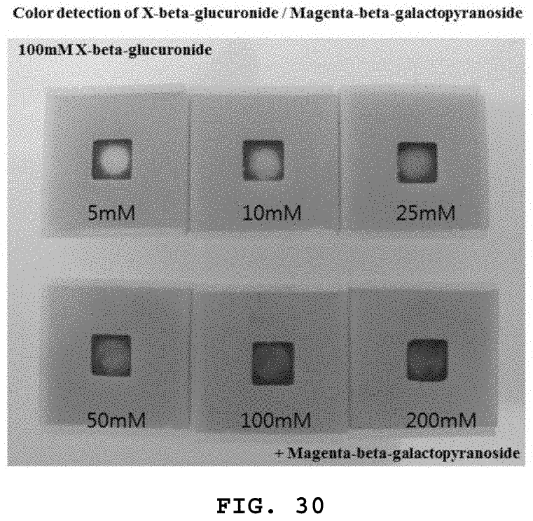

The results are shown in FIGS. 30 and 31.

As shown in FIGS. 30 and 31, the most appropriate mixture ratio of the two chromogenic reagents when detecting E. coli: O157 was determined to be 100 mM X-beta-glucuronide+10 mM Magenta-beta-galactopyranoside.

A very important factor in distinguishing Escherichia coli and Escherichia coli O157 based on color formation is that Escherichia coli is detected in blue by carrying out the coloring reaction to both reagents, and Escherichia coli O157, a food risk microorganism, is detected in purple, which is to easily distinguish and detect the two confusing microorganisms.

(D) Coloring Test of Microfluidic Paper Chip for E. coli: O517

Coloring test was performed on E. coli: O157 and other food risk microorganisms by performing a coloring test of microfluidic paper chip made with 100 mM X-beta-glucuronide and 10 mM Magenta-beta-galactopyranoside for detecting E. coli: O157.

To this end, each 5 μL of 100 mM X-beta-glucuronide was loaded on a paper that was prepared with pre-prepared patterns and was dried for 30 minutes in a 40° C. dryer. Afterwards 5 μL of 10 mM Magenta-beta-galactopyranoside was loaded on the same paper and was again dried for 30 minutes in a 40° C. dryer.

For the oxidation reagent used in preparing a microfluidic paper chip for detecting Escherichia coli O157, each 5 UL of 10 mM potassium ferriccyanide (K₃Fe(CN)₆) and potassium ferrocyanide (K₄Fe(CN)₆) was loaded as oxidation reagents on papers that were prepared with pre-prepared patterns and was dried for 30 minutes in a 40° C. dryer.

Furthermore, 5 UL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared microorganism suspension was injected thereto, reaction was carried out for 30 minutes at 37° C., and coloring reactions of paper-based microfluidic device for detecting Escherichia coli O157 were tested.

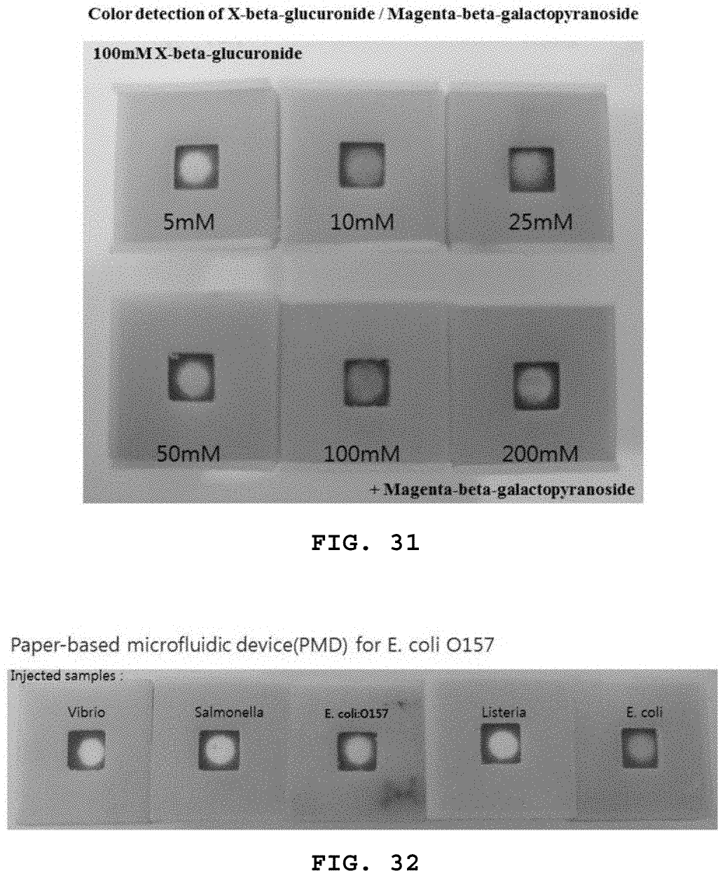

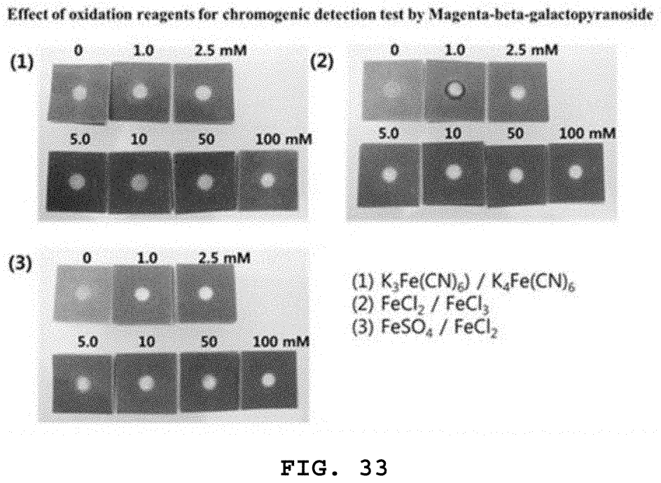

The results are shown in FIG. 32

As shown in FIG. 32, the pink coloring detection of interest for Escherichia coli O157 was identified, and in comparison, the blue coloring detection of interest for Escherichia coli was identified.

### Example 7

Evaluation on Detection of Vibrio vulnificus Using a Microfluidic Paper Chip

(A) Coloring Test of Microfluidic Paper Chip for Different Types and Concentrations of Oxidation Reagents

An investigation for an appropriate type and concentration of oxidation reagent was carried out according to the example 4 to prepare a microfluidic paper chip for detecting Vibrio vulnificus.

An investigation for a composition for developing an oxidation reagent was carried out to promote the oxidation of chromophore during a coloring reaction of the chromogenic reagent in detecting Vibrio vulnificus. To this end, 1.5 mL of Vibrio vulnificus cultured in said conditions was centrifuged, bacterial cells were collected, suspension was prepared by adding 0.5 mL of PBS, and was used as the sample.

Each 5 μL of potassium ferriccyanide (K₃Fe(CN)₆) and potassium ferrocyanide (K₄Fe(CN)₆) was loaded as oxidation reagents on papers that were prepared with pre-prepared patterns for different concentrations of FeCl₂ and FeCl₃, and FeSO₄ and FeCl₂, and was dried for 30 minutes in a 40° C. dryer.

Other than the oxidation reagents, each 5 μL of the lysis reagents prepared in said conditions that are necessary for the microfluidic paper chip assembly and the corresponding chromogenic reagents was loaded as oxidation reagents on papers that were prepared with pre-prepared patterns, and was dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), 50 μL of pre-prepared Vibrio vulnificus suspension was injected thereto, reaction was carried out for 30 minutes at 37° C., and coloring reactions for different types and concentrations of the oxidation reagents were tested.

The results are shown in FIG. 33.

As shown in FIG. 33, characteristics of oxidation reaction for the X-beta-glucopyranoside used for detecting Vibrio vulnificus were identified. Magenta-beta-galactopyranoside showed the best coloring reaction with 10 mM FeCl₂/FeCl₃.

(B) Coloring Test of Microfluidic Paper Chip for Different Types and Concentrations of Chromogenic Reagents

An investigation for an appropriate type and concentration of chromogenic reagent was carried out according to the example 3 to prepare a microfluidic paper chip for detecting Vibrio vulnificus.

An investigation for an optimized concentration of chromogenic reagent in coloring detection was carried out using X-beta-glucopyranoside as the chromogenic reagent in detecting Vibrio vulnificus. To this end, 1.5 mL of Vibrio vulnificus cultured in said conditions was centrifuged, bacterial cells were collected, suspension was prepared by adding 0.5 mL of PBS, and was used as the sample.

To investigate the optimized concentration of a chromogenic reagent for detecting Vibrio vulnificus, each 5 μL of 5, 10, 25, 50, 100, and 200 mM chromogenic reagents was loaded on papers that were prepared with pre-prepared patterns, and was dried for 30 minutes in a 40° C. dryer.

For the oxidation reagent used in preparing a microfluidic paper chip for detecting Vibrio vulnificus, each 5 μL of 10 mM FeCl₂ and FeCl₃ was loaded as oxidation reagents on papers that were prepared with pre-prepared patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared Vibrio vulnificus suspension was injected thereto, reaction was carried out for 30 minutes at 37° C., and coloring reactions for different types and concentrations of the chromogenic reagents were tested.

The results are shown in FIG. 34.

As shown in FIG. 34, characteristics of coloring reactions for different concentrations of X-beta-glucopyranoside in detecting Vibrio vulnificus were identified. It was identified that as the concentration of X-beta-glucopyranoside increased, the intensity of the coloring reaction increased. As the intensity of the coloring was similar at concentrations of 100 mM and higher, the concentration of X-beta-glucopyranoside is preferably 25-200 mM, and most preferably 100 mM.

(C) Coloring Test of Microfluidic Paper Chip for Vibrio vulnificus

Coloring test was performed on Vibrio vulnificus and other food risk microorganisms by performing a coloring test of microfluidic paper chip made with 100 mM X-beta-glucopyranoside for detecting Vibrio vulnificus.

To this end, 5 μL of 100 mM X-beta-glucopyranoside was loaded on a paper that was prepared with pre-prepared patterns and was dried for 30 minutes in a 40° C. dryer.

For the oxidation reagent used in preparing a microfluidic paper chip for detecting Vibrio vulnificus, each 5 μL of 10 mM potassium ferriccyanide (K₃Fe(CN)₆) and potassium ferrocyanide (K₄Fe(CN)₆) was loaded as oxidation reagents on papers that were prepared with patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared microorganism suspension was injected thereto, reaction was carried out for 30 minutes at 37° C., and coloring reactions of paper-based microfluidic device for detecting Vibrio vulnificus were tested.

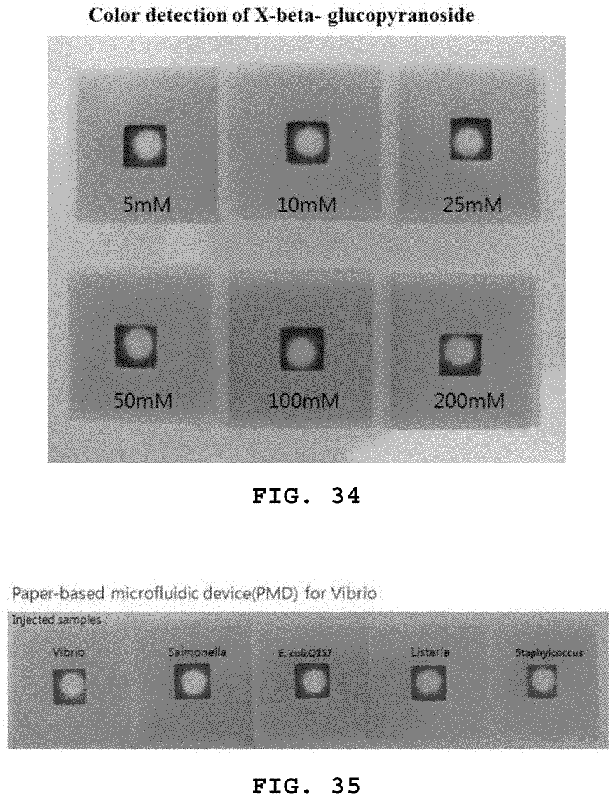

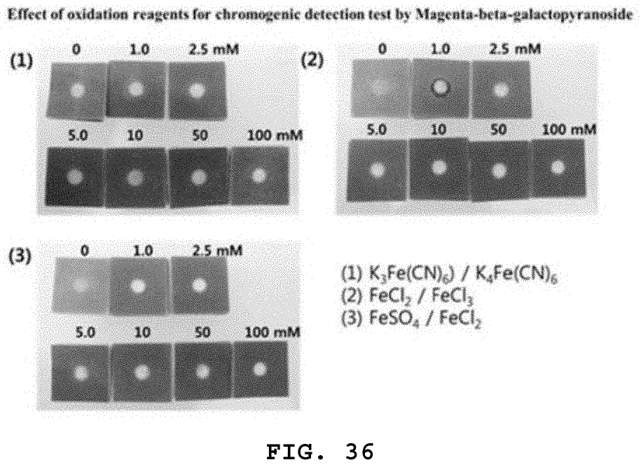

The results are shown in FIG. 35

As shown in FIG. 35, the light blue coloring detection of interest for Vibrio vulnificus was identified. The light blue coloring was also detected from Listeria monocytogenes, however, the growth of Listeria monocytogenes, a gram-positive bacterium, can be inhibited using an inhibitory factor that selectively inhibits gram-positive bacteria during enrichment culture, thus considered not to be problematic when detecting Vibrio vulnificus.

### Example 8

Evaluation on Detection of Salmonella Spp. Using a Microfluidic Paper Chip

(A) Coloring Test of Microfluidic Paper Chip for Different Types and Concentrations of Oxidation Reagents

An investigation for an appropriate type and concentration of oxidation reagent was carried out according to the example 4 to prepare a microfluidic paper chip for detecting Salmonella spp.

An investigation for a composition for developing an oxidation reagent was carried out to promote the oxidation of chromophore during a coloring reaction of the chromogenic reagent in detecting Salmonella spp. To this end, 1.5 mL of Salmonella spp. cultured in said conditions was centrifuged, bacterial cells were collected, suspension was prepared by adding 0.5 mL of PBS, and was used as the sample.

Each 5 μL of potassium ferriccyanide (K₃Fe(CN)₆) and potassium ferrocyanide (K₄Fe(CN)₆) was loaded as oxidation reagents on papers that were prepared with pre-prepared patterns for different concentrations of FeCl₂ and FeCl₃, and FeSO₄ and FeCl₂, and was dried for 30 minutes in a 40° C. dryer.

Other than the oxidation reagents, 5 μL of the lysis reagents prepared in said conditions that are necessary for the microfluidic paper chip assembly and the corresponding chromogenic reagents were loaded as oxidation reagents on papers that were prepared with pre-prepared patterns, and were dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), 50 μL of pre-prepared Salmonella spp. suspension was injected thereto, reaction was carried out for 30 minutes at 37° C., and coloring reactions for different types and concentrations of the oxidation reagents were tested.

The results are shown in FIG. 36.

As shown in FIG. 36, characteristics of oxidation reaction for the Salmone-alpha-glucopyranoside used for detecting Salmonella spp. were identified. No result of promoting a coloring reaction for an oxidation reagent was obtained in case of Salmone-alpha-glucopyranoside.

(B) Coloring Test of Microfluidic Paper Chip for Different Types and Concentrations of Chromogenic Reagents

An investigation for an appropriate type and concentration of chromogenic reagent was carried out according to the example 3 to prepare a microfluidic paper chip for detecting Salmonella spp.

An investigation for an optimized concentration of chromogenic reagent in coloring detection was carried out using Salmone-alpha-glucopyranoside and X-phosphate as the chromogenic reagent in detecting Salmonella spp. To this end, 1.5 mL of Salmonella spp. cultured in said conditions was centrifuged, bacterial cells were collected, suspension was prepared by adding 0.5 mL of PBS, and was used as the sample.

To investigate the optimized concentration of a chromogenic reagent for detecting Salmonella spp., each 5 μL of 5, 10, 25, 50, 100, and 200 mM chromogenic reagents was loaded on papers that were prepared with pre-prepared patterns, and was dried for 30 minutes in a 40° C. dryer.

For the oxidation reagent used in preparing a microfluidic paper chip for detecting Salmonella spp., each 5 μL of 10 mM potassium ferriccyanide (K₃Fe(CN)₆) and potassium ferrocyanide (K₄Fe(CN)₆) was loaded as oxidation reagents on papers that were prepared with pre-prepared patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared Salmonella spp. suspension was injected thereto, reaction was carried out for 30 minutes at 37° C., and coloring reactions for different types and concentrations of the chromogenic reagents were tested.

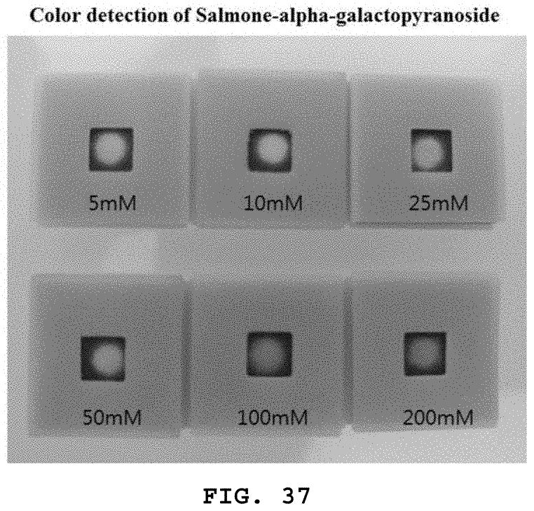

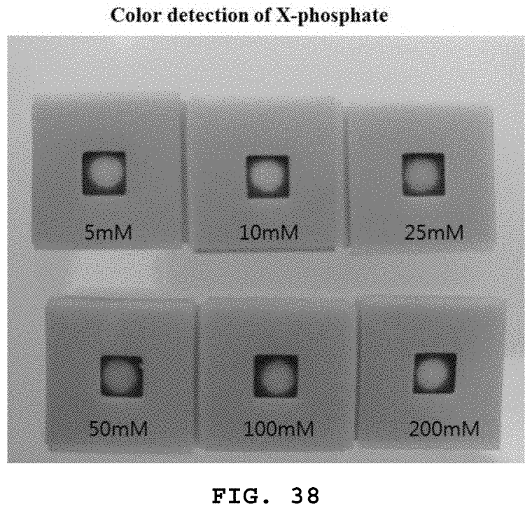

The results are shown in FIGS. 37 and 38.

As shown in FIG. 37, as a result of investigating the characteristics of coloring reactions for different concentrations of Salmone-alpha-glucopyranoside in detecting Salmonella spp., it was identified that as the concentration of Salmone-alpha-glucopyranoside increased, the intensity of the coloring reaction increased. As the best coloring detection was shown at 200 mM, the concentration of Salmone-alpha-glucopyranoside is preferably 25-300 mM, and most preferably 200 mM.

Furthermore, as shown in FIG. 38, as a result of investigating the coloring reaction for different concentrations of X-phosphate in detecting Salmonella spp., the degree of the coloring reaction increased as the concentration of X-phosphate increased, and the intensity of the coloring reaction rather decreased at 100 mM or higher concentration. Thus, the concentration of X-phosphate is preferably 25-100 mM, and most preferably 50 mM.

(C) Coloring Test of Microfluidic Paper Chip for Mixed Concentrations of Salmone-Alpha-Glucopyranoside and X-Phosphate

By referring to said results, the concentration mixture ratio of the two chromogenic reagents was investigated for an appropriate detection of Salmonella spp. To this end, 5 μL of 200 mM Salmone-alpha-glucopyranoside was loaded on a paper that was prepared with pre-prepared patterns and was dried for 30 minutes in a 40° C. dryer. Afterwards 5 μL of X-phosphate was mixed at different concentrations on the same paper and loaded on the paper that was prepared with pre-prepared patterns and was again dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), 50 μL of pre-prepared Salmonella spp. suspension was injected thereto, reaction was carried out for 30 minutes at 37° C., and coloring reactions for the mixture of the two chromogenic reagents were tested.

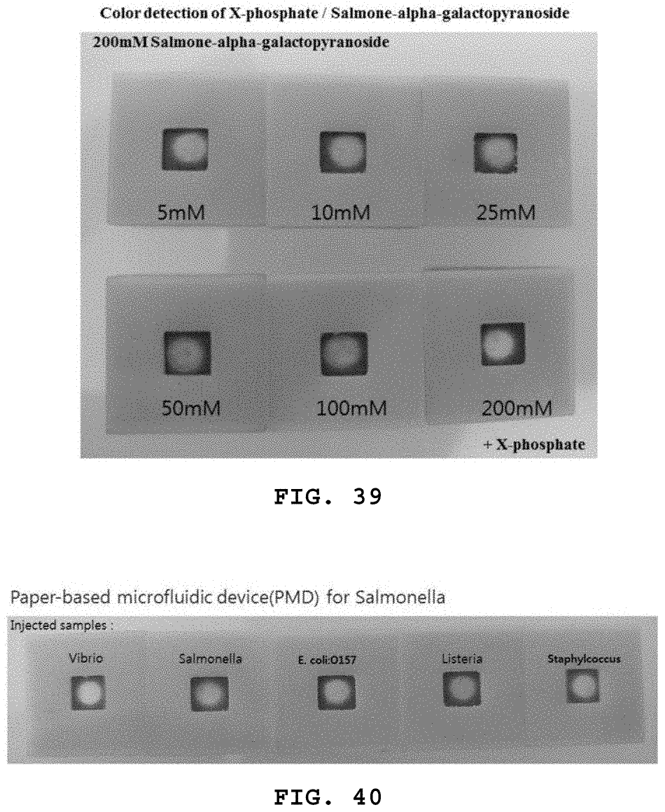

The results are shown in FIG. 39.

As shown in FIG. 39, the most appropriate mixture ratio of the two chromogenic reagents when detecting Salmonella spp. was determined to be 200 mM Salmone-alpha-glucopyranoside/50 mM X-phosphate.

Selective medium will have increased specificity by using two chromogenic substrates, but this is to distinguish and detect the Salmonella spp. more accurately by detecting in blue with double detection coloring reaction.

(D) Coloring Test of Microfluidic Paper Chip for Salmonella spp.

Coloring test was performed on Salmonella spp. and other food risk microorganisms by performing a coloring test of microfluidic paper chip made with 200 mM Salmone-alpha-glucopyranoside and 50 mM X-phosphate for detecting Salmonella spp.

To this end, 5 μL of 200 mM Salmone-alpha-glucopyranoside was loaded on a paper that was prepared with pre-prepared patterns and was dried for 30 minutes in a 40° C. dryer. Afterwards 5 μL of 50 mM X-phosphate was loaded on the same paper and was again dried for 30 minutes in a 40° C. dryer.

For the oxidation reagent used in preparing a microfluidic paper chip for detecting Salmonella spp., each 5 μL of 10 mM potassium ferriccyanide (K₃Fe(CN)₆) and potassium ferrocyanide (K₄Fe(CN)₆) was loaded as oxidation reagents on papers that were prepared with pre-prepared patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared microorganism suspension was injected thereto, reaction was carried out for 30 minutes at 37° C., and coloring reactions of paper-based microfluidic device for detecting Salmonella spp. were tested.

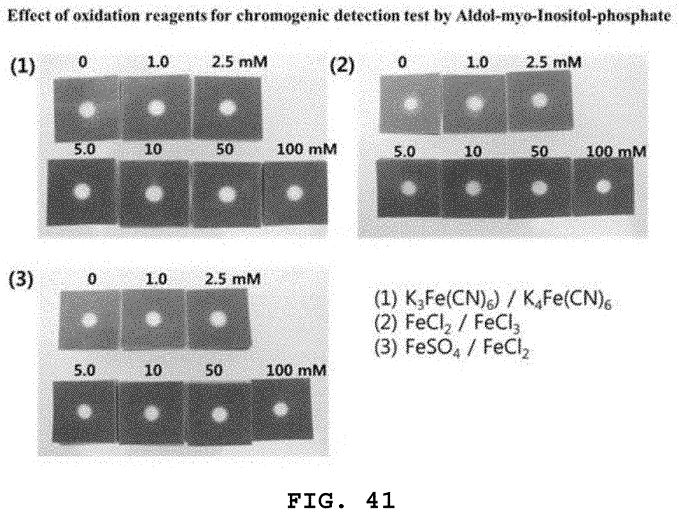

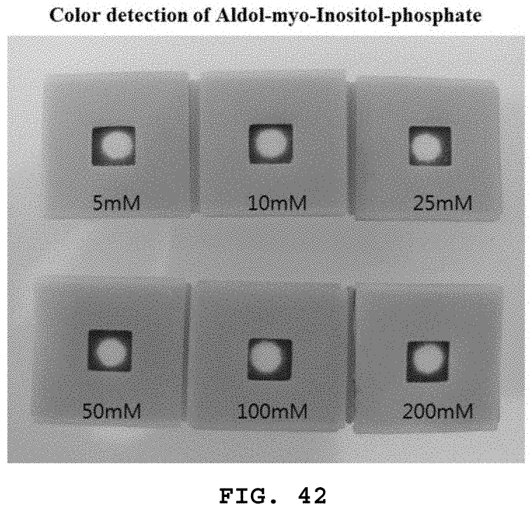

The results are shown in FIG. 40

As shown in FIG. 40, the pink coloring detection of interest for Salmonella spp. was identified, and in cases of other bacteria for comparison, no coloring detection for Vibrio vulnificus and Staphylococcus aureus was identified, and the pink coloring detection for Escherichia coli O157 and Listeria monocytogenes was identified.

### Example 9

Evaluation on Detection of Listeria monocytogenes Using a Microfluidic Paper Chip

(A) Coloring Test of Microfluidic Paper Chip for Different Types and Concentrations of Oxidation Reagents

An investigation for an appropriate type and concentration of oxidation reagent was carried out according to the example 4 to prepare a microfluidic paper chip for detecting Listeria monocytogenes.

An investigation for a composition for developing an oxidation reagent was carried out to promote the oxidation of chromophore during a coloring reaction of the chromogenic reagent in detecting Listeria monocytogenes. To this end, 1.5 mL of Listeria monocytogenes cultured in said conditions was centrifuged, bacterial cells were collected, suspension was prepared by adding 0.5 mL of PBS, and was used as the sample.

Each 5 μL of potassium ferriccyanide (K₃Fe(CN)₆) and potassium ferrocyanide (K₄Fe(CN)₆) was loaded as oxidation reagents on papers that were prepared with pre-prepared patterns for different concentrations of FeCl₂ and FeCl₃, and FeSO₄ and FeCl₂, and was dried for 30 minutes in a 40° C. dryer.

Other than the oxidation reagents, 5 μL of the lysis reagents prepared in said conditions that are necessary for the microfluidic paper chip assembly and the corresponding chromogenic reagents was loaded as oxidation reagents on papers that were prepared with pre-prepared patterns, and was dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), 50 μL of pre-prepared Listeria monocytogenes suspension was injected thereto, reaction was carried out for 30 minutes at 37° C., and coloring reactions for different types and concentrations of the oxidation reagents were tested.

The results are shown in FIG. 41.

As shown in FIG. 36, characteristics of oxidation reaction for the Aldol-myo-inositol-phosphate used for detecting Listeria monocytogenes were identified. Aldol-myo-inositol-phosphate showed the best coloring reaction with 10 mM FeCl₂/FeCl₃.

(B) Coloring test of microfluidic paper chip for different types and concentrations of chromogenic reagents

An investigation for an appropriate type and concentration of chromogenic reagent was carried out according to the example 3 to prepare a microfluidic paper chip for detecting Listeria monocytogenes.

An investigation for an optimized concentration of chromogenic reagent in coloring detection was carried out using Aldol-myo-inositol-phosphate as the chromogenic reagent in detecting Listeria monocytogenes. To this end, 1.5 mL of Listeria monocytogenes cultured in said conditions was centrifuged, bacterial cells were collected, suspension was prepared by adding 0.5 mL of PBS, and was used as the sample.

To investigate the optimized concentration of a chromogenic reagent for detecting Listeria monocytogenes, each 5 μL of 5, 10, 25, 50, 100, and 200 mM chromogenic reagents was loaded on papers that were prepared with pre-prepared patterns, and was dried for 30 minutes in a 40° C. dryer.

For the oxidation reagent used in preparing a microfluidic paper chip for detecting Listeria monocytogenes, each 5 μL of 10 mM FeCl₂ and FeCl₃ was loaded as oxidation reagents on papers that were prepared with pre-prepared patterns and was dried for 30 minutes in a 40° C. dryer.

Furthermore, 5 UL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared Listeria monocytogenes suspension was injected thereto, reaction was carried out for 30 minutes at 37° C., and coloring reactions for different types and concentrations of the chromogenic reagents were tested.

The results are shown in FIG. 42.

As shown in FIG. 42, characteristics of coloring reactions for different concentrations of Aldol-myo-inositol-phosphate in detecting Listeria monocytogenes were identified. It was identified that as the concentration of Aldol-myo-inositol-phosphate increased, the degree of the coloring reaction rapidly decreased.

Meanwhile, as the coloring appeared at concentrations of 10 mM or lower, the coloring reaction tests were reinvestigated for concentrations of 10 mM or lower to find the optimized concentration of Aldol-myo-inositol-phosphate in more detail.

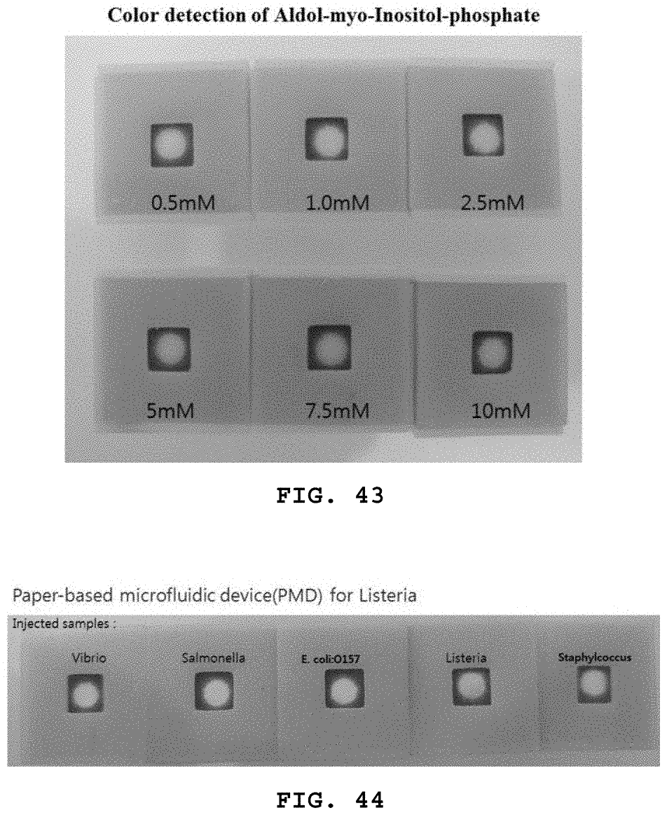

The results are shown in FIG. 43.

As shown in FIG. 43, as a result of having reinvestigated the coloring reaction tests for concentrations of 10 mM or lower to find the optimized concentration of Aldol-myo-inositol-phosphate in more detail, the concentration of Aldol-myo-inositol-phosphate is preferably 1-10 mM, and most preferably 7.5 mM.

(C) Coloring Test of Microfluidic Paper Chip for Listeria monocytogenes

Coloring test was performed on Listeria monocytogenes and other food risk microorganisms by performing a coloring test of microfluidic paper chip made with 7.5 mM Aldol-myo-inositol-phosphate for detecting Listeria monocytogenes.

To this end, 5 μL of 7.5 mM Aldol-myo-inositol-phosphate was loaded on a paper that was prepared with pre-prepared patterns and was dried for 30 minutes in a 40° C. dryer.

For the oxidation reagent used in preparing a microfluidic paper chip for detecting Salmonella spp., each 5 μL of 10 mM FeCl₂ and FeCl₃ was loaded as oxidation reagents on papers that were prepared with pre-prepared patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared microorganism suspension was injected thereto, reaction was carried out for 30 minutes at 37° C., and coloring reactions of paper-based microfluidic device for detecting Listeria monocytogenes were tested.

The results are shown in FIG. 44

As shown in FIG. 44, the pink coloring detection of interest for Listeria monocytogenes was identified, and no coloring detection was identified for other bacteria as aimed.

### Example 10

Evaluation on Detection of Staphylococcus aureus Using a Microfluidic Paper Chip

(A) Coloring Test of Microfluidic Paper Chip for Different Types and Concentrations of Oxidation Reagents

An investigation for an appropriate type and concentration of oxidation reagent was carried out according to the example 4 to prepare a microfluidic paper chip for detecting Staphylococcus aureus.

An investigation for a composition for developing an oxidation reagent was carried out to promote the oxidation of chromophore during a coloring reaction of the chromogenic reagent in detecting Staphylococcus aureus. To this end, 1.5 mL of Staphylococcus aureus cultured in said conditions was centrifuged, bacterial cells were collected, suspension was prepared by adding 0.5 mL of PBS, and was used as the sample.

Each 5 μL of potassium ferriccyanide (K₃Fe(CN)₆) and potassium ferrocyanide (K₄Fe(CN)₆) was loaded as oxidation reagents on papers that were prepared with pre-prepared patterns for different concentrations of FeCl₂ and FeCl₃, and FeSO₄ and FeCl₂, and was dried for 30 minutes in a 40° C. dryer.

Other than the oxidation reagents, 5 μL of the lysis reagents prepared in said conditions that are necessary for the microfluidic paper chip assembly and the corresponding chromogenic reagents was loaded as oxidation reagents on papers that were prepared with pre-prepared patterns, and was dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), 50 μL of pre-prepared Staphylococcus aureus suspension was injected thereto, reaction was carried out for 30 minutes at 37° C., and coloring reactions for different types and concentrations of the oxidation reagents were tested.

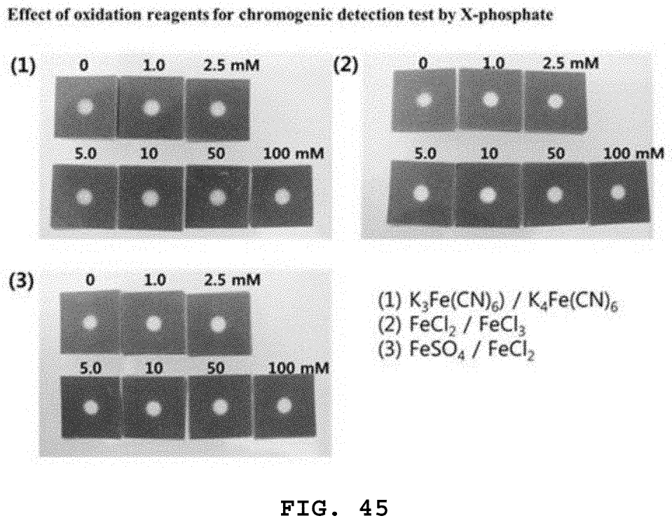

The results are shown in FIG. 45.

As shown in FIG. 45, characteristics of oxidation reaction for the X-phosphate used for detecting Staphylococcus aureus were identified. X-phosphate showed the best coloring reaction with 10 mM potassium ferriccyanide (K₃Fe(CN)₆) and potassium ferrocyanide (K₄Fe(CN)₆).

(B) Coloring Test of Microfluidic Paper Chip for Different Types and Concentrations of Chromogenic Reagents

An investigation for an appropriate type and concentration of chromogenic reagent was carried out according to the example 3 to prepare a microfluidic paper chip for detecting Staphylococcus aureus.

An investigation for an optimized concentration of chromogenic reagent in coloring detection was carried out using Magenta-beta-galactopyranoside and X-phosphate as the chromogenic reagent in detecting Staphylococcus aureus. To this end, 1.5 mL of Staphylococcus aureus cultured in said conditions was centrifuged, bacterial cells were collected, suspension was prepared by adding 0.5 mL of PBS, and was used as the sample.

To investigate the optimized concentration of a chromogenic reagent for detecting Staphylococcus aureus, each 5 μL of 5, 10, 25, 50, 100, and 200 mM chromogenic reagents was loaded on papers that were prepared with pre-prepared patterns, and was dried for 30 minutes in a 40° C. dryer.

For the oxidation reagent used in preparing a microfluidic paper chip for detecting Staphylococcus aureus, each 5 μL of 10 mM potassium ferriccyanide (K₃Fe(CN)₆) and potassium ferrocyanide (K₄Fe(CN)₆) was loaded as oxidation reagents on papers that were prepared with pre-prepared patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 μL of pre-prepared Staphylococcus aureus suspension was injected thereto, reaction was carried out for 30 minutes at 37° C., and coloring reactions for different types and concentrations of the chromogenic reagents were tested.

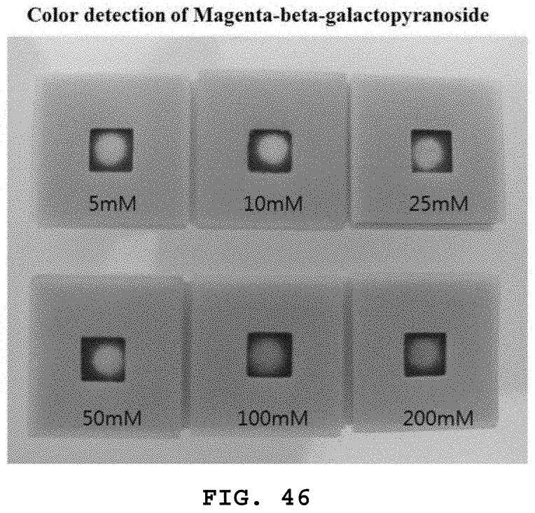

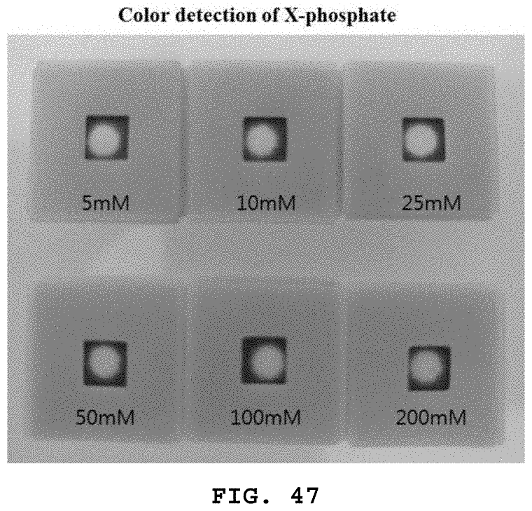

The results are shown in FIGS. 46 and 47.

As shown in FIG. 46, the characteristics of coloring reactions for different concentrations of Magenta-beta-galactopyranoside in detecting Staphylococcus aureus were identified. It was identified that as the concentration of Magenta-beta-galactopyranoside increased, the degree of the coloring reaction increased. As the best coloring detection was shown at the Magenta-beta-galactopyranoside concentration of 100 mM or higher, the optimum concentration of Magenta-beta-galactopyranoside was set to be 100 mM.

Furthermore, as shown in FIG. 47, as a result of investigating the coloring reaction for different concentrations of X-phosphate in detecting Staphylococcus aureus, the degree of the coloring reaction increased as the concentration of X-phosphate increased, and the intensity of the coloring reaction rather decreased at 100 mM or higher concentration. Thus, the concentration of X-phosphate is preferably 25-100 mM, and most preferably 50 mM.

(C) Coloring Test of Microfluidic Paper Chip for Mixed Concentrations of Magenta-Beta-Galactopyranoside and X-Phosphate

By referring to said results, the concentration mixture ratio of the two chromogenic reagents was investigated for an appropriate detection of Salmonella spp. To this end, 5 μL of 100 mM Magenta-beta-galactopyranoside was loaded on a paper that was prepared with pre-prepared patterns and was dried for 30 minutes in a 40° C. dryer. Afterwards 5 μL of X-phosphate was mixed at different concentrations on the same paper and loaded on the paper that was prepared with pre-prepared patterns and was again dried for 30 minutes in a 40° C. dryer.

Each paper was laminated in the order of the first layer (inlet)—the second layer (lysis reagent)—the third layer (oxidation)—the fourth layer (chromogenic reagent)—the fifth layer (outlet), 50 μL of pre-prepared Staphylococcus aureus suspension was injected thereto, reaction was carried out for 30 minutes at 37° C., and coloring reactions for the mixture of the two chromogenic reagents were tested.

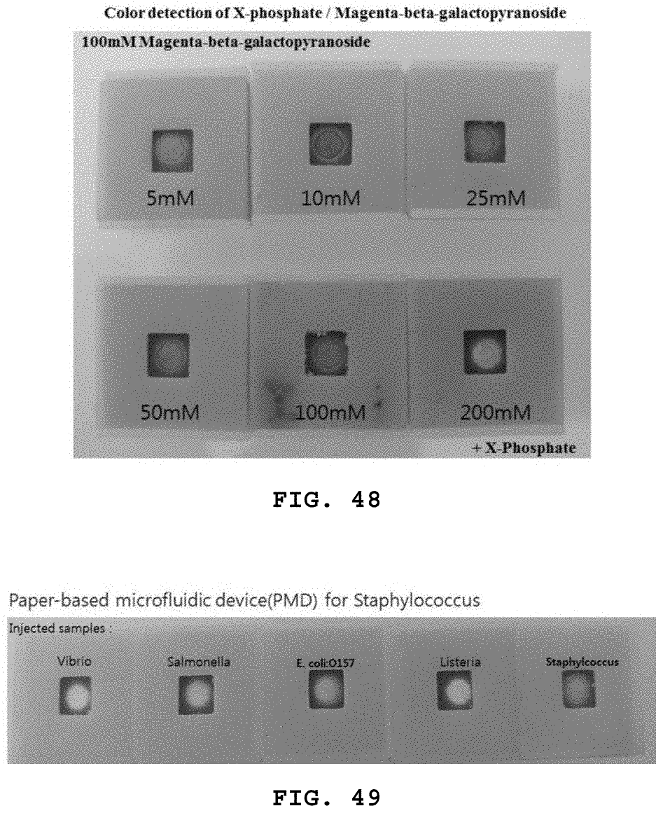

The results are shown in FIG. 48.

As shown in FIG. 48, the most appropriate mixture ratio of the two chromogenic reagents when detecting Staphylococcus aureus was determined to be 100 mM Magenta-beta-galactopyranoside/25 mM X-phosphate.

Selective medium will have increased specificity by using two chromogenic substrates, but this is to distinguish and detect the Staphylococcus aureus more accurately by detecting in blue with double detection coloring reaction.

(D) Coloring Test of Microfluidic Paper Chip for Staphylococcus aureus

Coloring test was performed on Staphylococcus aureus and other food risk microorganisms by performing a coloring test of microfluidic paper chip made with 100 mM Magenta-beta-galactopyranoside and 25 mM X-phosphate for detecting Staphylococcus aureus.

To this end, 5 μL of 100 mM Magenta-beta-galactopyranoside was loaded on a paper that was prepared with pre-prepared patterns and was dried for 30 minutes in a 40° C. dryer. Afterwards 5 μL of 25 mM X-phosphate was loaded on the paper and was again dried for 30 minutes in a 40° C. dryer.

For the oxidation reagent used in preparing a microfluidic paper chip for detecting Staphylococcus aureus, each 5 μL of 10 mM potassium ferriccyanide (K₃Fe(CN)₆) and potassium ferrocyanide (K₄Fe(CN)₆) was loaded as oxidation reagents on papers that were prepared with pre-prepared patterns and was dried for 30 minutes in a 40° C. dryer.

Furthermore, 5 μL of the lysis reagents developed in said conditions needed for microfluidic paper chip assembly was each loaded on papers that were prepared with pre-prepared patterns and was dried for 30 minutes in a 40° C. dryer.

After laminating each paper in the order of the first layer (inlet)—the second layer (lysis reagent)—the third layer (oxidation)—the fourth layer (chromogenic reagent)—the fifth layer (outlet), 50 UL of pre-prepared microorganism suspension was injected thereto, reaction was carried out for 30 minutes at 37° C., and coloring reactions of paper-based microfluidic device for detecting Staphylococcus aureus were tested.

The results are shown in FIG. 49

As shown in FIG. 49, the pink and blue coloring detection of interest for Staphylococcus aureus was identified, and in cases of other bacteria for comparison, no coloring detection for Vibrio vulnificus and Listeria monocytogenes was identified, and the pink coloring detection for Escherichia coli O157 and light blue coloring detection for Salmonella spp. were identified.

The aforementioned preferred embodiments of the present invention are disclosed to solve technical tasks, and it will be apparent to those skilled in this art that various modifications, variations and additions can be made thereto without departing from the spirit and scope of the present invention, and such modifications, variations and the like should be construed to be included in the following claims.

1. A microfluidic paper chip for detecting a microorganism, the microfluidic paper chip comprising a lysis layer and a chromogenic layer which are laminated in this order, wherein
the lysis layer is composed of a hydrophilic paper containing a lysis reagent composition and
the chromogenic layer is composed of a hydrophilic paper containing a chromogenic reagent.

2. The microfluidic paper chip of claim 1, further comprising an outer layer laminated on the lysis layer or under the chromogenic layer, wherein the outer layer is composed of a hydrophilic paper.

3. The microfluidic paper chip of claim 1, further comprising an oxidation layer provided between the lysis layer and the chromogenic layer, wherein the oxidation layer is composed of a hydrophilic paper containing an oxidation reagent.

4. The microfluidic paper chip of claim 1, wherein a fluidic channel is formed by printing a hydrophobic material on edges of the hydrophilic paper to form a barrier wall.

5. The microfluidic paper chip of claim 1, wherein the hydrophilic paper is a chromatography paper or a filter paper.

6. The microfluidic paper chip of claim 1, wherein the microorganism is at least one selected from the group consisting of Salmonella, Bacillus, Listeria, Vibrio, Campylobacter, Staphylococcus aureus, Escherichia Coliform, E. coli, Shigella, Legionella, Enterobacter sakazakii, Citrobacter, Proteus, Methicillin-resistant Staphylococcus aureus (MRSA), and E. coli O157.

7. The microfluidic paper chip of claim 1, wherein the lysis reagent composition is at least one selected from the group consisting of Tergitol NP-9, Tergitol NP-10, Tergitol NP-40, Triton X-100, Tween 80, BMT, SB3-8, SB3-10, SB3-14, and SB3-16.

8. The microfluidic paper chip of claim 7, wherein the lysis reagent composition further comprises C7BzO (3-[[3-(4-heptylphenyl)-3-hydroxypropyl]-dimethylazaniumyl]propane-1-sulfonate).

9. The microfluidic paper chip of claim 8, wherein the lysis reagent composition further comprises a silica bead.

10. The microfluidic paper chip of claim 1, wherein the chromogenic reagent is at least one selected from the group consisting of 5-bromo-4-chloro-3-indoxyl-beta-L-arabinopyranoside, 5-bromo-4-chloro-3-indoxyl-beta-D-glucuronic acid, 5-bromo-4-Chloro-3-indoxyl-alpha-D-maltotrioside, 5-bromo-4-chloro-3-indoxyl-N-acetyl-beta-D-galactosamide, 5-bromo-4-Chloro-3-indoxyl-N-acetyl-beta-D-glucosaminid, 5-bromo-4-chloro-3-indoxyl-N-acetyl-beta-D-galactosam ide, 5-Bromo-4-chloro-3-indoxyl-alpha-D-N-acetylneuraminic acid, 5-bromo-4-chloro-3-indoxyl-alpha-L-araminofuranoside, 5-bromo-4-Chloro-3-indoxyl-beta-D-cellobioside, 5-bromo-4-chloro-3-indoxyl-choline phosphate, 5-bromo-4-chloro-3-indoxyl-alpha-D-fucopyranoside, 5-bromo-4-chloro-3-indoxyl-alpha-L-fucoparinoside, 5-bromo-4-chloro-3-indoxyl-alpha-D-galactopyranoside, 5-bromo-4-chloro-3-indoxyl-beta-D-galactopyranoside, 5-bromo-4-chloro-3-indoxyl-alpha-D-glucopyranoside, 5-bromo-4-chloro-3-indoxyl-beta-D-glucopyranoside, 5-bromo-4-chloro-3-indoxyl-myo-inositol-1-phosphate, 5-bromo-4-chloro-3-indoxyl-alpha-D-mannopyranoside, 5-bromo-4-chloro-3-indoxyl-beta-D-mannopyranoside, 5-bromo-4-chloro-3-indoxyl-alpha-D-xylopyranoside, 5-bromo-4-chloro-3-indoxyl butylate, 5-bromo-4-chloro-3-indoxyl caprylate, 5-Bromo-4-chloro-3-indoxyl nonanonate, 5-bromo-4-chloro-3-indoxyl oleate, 5-bromo-4-chloro-3-indoxyl palmitate, 5-Bromo-4-chloro-3-indoxyl phosphate, 5-bromo-4-chloro-3-indoxyl sulfate, 5-bromo-4-chloro-3-indoxyl-1-acetate, 5-bromo-4-chloro-3-indoxyl-3-acetate, 6-chloro-3-indoxyl-N-acetyl-beta-D-glucosaminide, 6-chloro-3-indoxyl-alpha-D-mannopyranoside, 6-chloro-3-indoxyl-beta-D-mannopyranoside, 6-chloro-3-Indoxyl-myo-inositol-1-phosphate, 6-chloro-3-indoxyl-N-acetyl-beta-D-galactosaminide, 6-chloro-3-indoxyl-beta-D-cellobioside, 6-chloro-3-indoxyl-alpha-D-galactopyranoside, 6-chloro-3-indoxyl-beta-D-galactopyranoside, 6-chloro-3-indoxyl-alpha-D-glucopyranoside, 6-chloro-3-indoxyl-beta-D-glucopyranoside, 6-chloro-3-indoxyl-beta-D-glucuronic acid, 6-chloro-3-indoxyl butylate, 6-Chloro-3-indoxyl caprylate, 6-Chloro-3-indoxyl nonanoate, 6-Chloro-3-indoxyl oleate, 6-Chloro-3-indoxyl palmitate, 6-chloro-3-indoxyl phosphate, 6-chloro-3-indoxyl sulfate, 6-chloro-3-indoxyl-1-acetate, 5-bromo-6-chloro-3-indoxyl-N-acetyl-beta-D-glucosaminide, 5-bromo-6-chloro-3-indoxyl-beta-D-fucopyranoside, 5-bromo-6-chloro-3-indoxyl-alpha-D-galactopyranoside, 5-bromo-6-chloro-3-indoxyl-beta-D-galactopyranoside, 5-bromo-6-chloro-3-indoxyl-alpha-D-glucopyranoside, 5-bromo-6-chloro-3-indoxyl-beta-D-glucuronic acid, 5-bromo-6-chloro-3-indoxyl-alpha-D-glucopyranoside, 5-bromo-6-chloro-3-indoxyl-myo-inositol-1-phosphate, 5-bromo-6-chloro-3-indoxyl butylate, 5-bromo-6-chloro-3-indoxyl caprylate, 5-bromo-6-chloro-3-indoxyl nonanonate, 5-bromo-6-chloro-3-indoxyl palmitate, 5-bromo-6-chloro-3-indoxyl choline phosphate, 5-bromo-6-chloro-3-indoxyl phosphate, 5-bromo-6-chloro-3-indoxyl sulfate, 5-bromo-6-chloro-3-indoxyl-3-acetate, Aldol 518 beta-D-galactopyranoside, Aldol 518 alpha-D-galactopyranoside, Aldol 518 alpha-D-glucopyranoside, Aldol 518 beta-D-glucopyranoside, Aldol 518 beta-D-glucuronic acid, Aldol 518 myo-inositol-1-phosphate, Aldol 515 caprylate, Aldol 515 palmitate, Aldol 515 phosphate and Aldol 515 acetate.

11. The microfluidic paper chip of claim 3, wherein the oxidation reagent is at least one selected from the group consisting of a mixture of potassium ferriccyanide (K₃Fe(CN)₆) and potassium ferrocyanide (K₄Fe(CN)₆), a mixture of FeCl₂ and FeCl₃, and a mixture of FeSO₄ and FeCl₂.

12. A method for producing a microfluidic paper chip, the method comprising:
(a) a step of forming a hydrophobic barrier wall by printing a hydrophobic material on the edges of multiple papers made of a hydrophilic material;
(b) a step of making a lysis reagent composition absorbed into a hydrophilic area of one of the papers on which the hydrophobic material is printed and then performing drying;
(c) a step of making a chromogenic reagent absorbed into a hydrophilic area of another one of the papers on which the hydrophobic material is printed and then performing drying; and
(d) a step of sequentially laminating the paper on which the hydrophobic material is printed, the paper on which said lysis reagent composition is printed, the paper on which said chromogenic reagent is printed, and the paper on which said hydrophobic material is printed.

13. A method of detecting a microorganism by using the microfluidic paper chip for detecting a microorganism according to claim 1.
